SEQUENCE LISTING (45 sequences)

SEQ ID NO: 1 - protein, 15 aa, Artificial Sequence
  Hcrt1-15
MNLPSTKVSWAAVTL

SEQ ID NO: 2 - protein, 15 aa, Artificial Sequence
  Hcrt5-19
STKVSWAAVTLLLLL

SEQ ID NO: 3 - protein, 15 aa, Artificial Sequence
  Hcrt9-23
SWAAVTLLLLLLLLP

SEQ ID NO: 4 - protein, 15 aa, Artificial Sequence
  Hcrt13-27
VTLLLLLLLLPPALL

SEQ ID NO: 5 - protein, 15 aa, Artificial Sequence
  Hcrt17-31
LLLLLLPPALLSSGA

SEQ ID NO: 6 - protein, 15 aa, Artificial Sequence
  Hcrt21-35
LLPPALLSSGAAAQP

SEQ ID NO: 7 - protein, 15 aa, Artificial Sequence
  Hcrt25-39
ALLSSGAAAQPLPDC

SEQ ID NO: 8 - protein, 15 aa, Artificial Sequence
  Hcrt29-43
SGAAAQPLPDCCRQK

SEQ ID NO: 9 - protein, 15 aa, Artificial Sequence
  Hcrt33-47
AQPLPDCCRQKTCSC

SEQ ID NO: 10 - protein, 15 aa, Artificial Sequence
  Hcrt37-51
PDCCRQKTCSCRLYE

SEQ ID NO: 11 - protein, 15 aa, Artificial Sequence
  Hcrt41-55
RQKTCSCRLYELLHG

SEQ ID NO: 12 - protein, 15 aa, Artificial Sequence
  Hcrt45-59
CSCRLYELLHGAGNH

SEQ ID NO: 13 - protein, 15 aa, Artificial Sequence
  Hcrt49-63
LYELLHGAGNHAAGI

SEQ ID NO: 14 - protein, 15 aa, Artificial Sequence
  Hcrt53-67
LHGAGNHAAGILTLG

SEQ ID NO: 15 - protein, 15 aa, Artificial Sequence
  Hcrt57-71
GNHAAGILTLGKRRS

SEQ ID NO: 16 - protein, 15 aa, Artificial Sequence
  Hcrt61-75
AGILTLGKRRSGPPG

SEQ ID NO: 17 - protein, 15 aa, Artificial Sequence
  Hcrt65-79
TLGKRRSGPPGLQGR

SEQ ID NO: 18 - protein, 15 aa, Artificial Sequence
  Hcrt69-83
RRSGPPGLQGRLQRL

SEQ ID NO: 19 - protein, 15 aa, Artificial Sequence
  Hcrt73-87
PPGLQGRLQRLLQAS

SEQ ID NO: 20 - protein, 15 aa, Artificial Sequence
  Hcrt77-91
QGRLQRLLQASGNHA

SEQ ID NO: 21 - protein, 15 aa, Artificial Sequence
  Hcrt81-95
QRLLQASGNHAAGIL

SEQ ID NO: 22 - protein, 15 aa, Artificial Sequence
  Hcrt85-99
QASGNHAAGILTMGR

SEQ ID NO: 23 - protein, 15 aa, Artificial Sequence
  Hcrt89-103
NHAAGILTMGRRAGA

SEQ ID NO: 24 - protein, 15 aa, Artificial Sequence
  Hcrt93-107
GILTMGRRAGAEPAP

SEQ ID NO: 25 - protein, 15 aa, Artificial Sequence
  Hcrt97-111
MGRRAGAEPAPRPCL

SEQ ID NO: 26 - protein, 15 aa, Artificial Sequence
  Hcrt101-115
AGAEPAPRPCLGRRC

SEQ ID NO: 27 - protein, 15 aa, Artificial Sequence
  Hcrt105-119
PAPRPCLGRRCSAPA

SEQ ID NO: 28 - protein, 15 aa, Artificial Sequence
  Hcrt109-123
PCLGRRCSAPAAASV

SEQ ID NO: 29 - protein, 15 aa, Artificial Sequence
  Hcrt113-127
RRCSAPAAASVAPGG

SEQ ID NO: 30 - protein, 15 aa, Artificial Sequence
  Hcrt117-131
APAAASVAPGGQSGI

SEQ ID NO: 31 - protein, 9 aa, Artificial Sequence
  HCRT 1-15 core
LPSTKVSWA

SEQ ID NO: 32 - protein, 9 aa, Artificial Sequence
  HCRT 25-39 core
SSGAAAQPL

SEQ ID NO: 33 - protein, 9 aa, Artificial Sequence
  HCRT 56-69 core
NHAAGILTL

SEQ ID NO: 34 - protein, 9 aa, Artificial Sequence
  HCRT 87-100 core
NHAAGILTM

SEQ ID NO: 35 - protein, 131 aa, Homo sapiens
  ref <308>: DATABASE ACCESSION NUMBER: NCBI/NP_001515
  ref <309>: DATABASE ENTRY DATE: 2018-12-02
  ref <313>: RELEVANT RESIDUES IN SEQ ID NO: (1)..(131)
MNLPSTKVSWAAVTLLLLLLLLPPALLSSGAAAQPLPDCCRQKTCSCRLYELLHGAGNHAAGILTLGKRRSGPPGLQGRLQRLLQASGNHAAGILTMGRRAGAEPAPRPCLGRRCSAPAAASVAPGGQSGI

SEQ ID NO: 36 - protein, 15 aa, Homo sapiens
  misc_feature (5)..(5): Xaa can be any naturally occurring amino acid
CSVEXDRGRSETQYF

SEQ ID NO: 37 - protein, 11 aa, Artificial Sequence
  HCRT87-97-NH2
SGNHAAGILTM

SEQ ID NO: 38 - protein, 15 aa, Artificial Sequence
  CLIP87-101
PVSKMRMATPLLMQA

SEQ ID NO: 39 - protein, 15 aa, Artificial Sequence
  thrombin-cleavable linker
GGGGSLVPRGSGGGG

SEQ ID NO: 40 - protein, 13 aa, Artificial Sequence
  soluble DQ6-HCRT1-13
MNLPSTKVSWAAV

SEQ ID NO: 41 - protein, 13 aa, Artificial Sequence
  DQ6-HCRT25-37
ALLSSGAAAQPLP

SEQ ID NO: 42 - protein, 14 aa, Artificial Sequence
  DQ6-HCRT56-69
AGNHAAGILTLGKR

SEQ ID NO: 43 - protein, 14 aa, Artificial Sequence
  DQ6-HCRT87-100
SGNHAAGILTMGRR

SEQ ID NO: 44 - protein, 14 aa, Artificial Sequence
  EBV486-499
RALLARSHVERTTD

SEQ ID NO: 45 - protein, 18 aa, Artificial Sequence
  EBV486-500
GGGRALLARSHVERTTDE

(19) United States
(12) Patent Application Publication
(10) Pub. No.: US 2023/0160890 A1
(43) Pub. Date: May 25, 2023

(54) COMPOSITIONS AND METHODS FOR DIAGNOSING NARCOLEPSY

(71) Applicant: The Board of Trustees of the Leland Stanford Junior University, Stanford, CA (US)

(72) Inventors: Wei Jiang, Stanford, CA (US); Elizabeth Mellins, Stanford, CA (US)

(21) Appl. No.: 17/414,829

(22) PCT Filed: Dec. 18, 2019
(86) PCT No.: PCT/US2019/067168

§ 371 Date: Jun. 16, 2021

Related U.S. Application Data
(60) Provisional application No. 62/781,876, filed on Dec. 19, 2018.

(51) Int. Cl. G01N 33/569; G01N 33/68

(52) U.S. Cl. CPC G01N 33/56972; G01N 33/686; G01N 2800/2864

(57) ABSTRACT

Compositions, methods, and kits are provided for diagnosing narcolepsy. Susceptibility to type 1 narcolepsy is linked with the human leukocyte antigen (HLA)-DQ6 allele and a single nucleotide polymorphism (SNP) in the T cell receptor gene segment TRAJ24. The presence of T cells reactive with hypocretin (HCRT) autoantigens is also associated with development of narcolepsy. Compositions and methods are provided for detecting autoreactive T cells specific for hypocretin (HCRT) autoantigens presented by HLA-DQ6.

### BACKGROUND

With a prevalence of 25-50/100,000 people (Longstreth et al. Sleep 30, 13-26 (2007)), type 1 narcolepsy (T1N) with cataplexy is a sleep disorder that currently lacks a cure. It results from the loss of hypocretin (HCRT)-producing neurons in the hypothalamus, causing an undetectable level of HCRT in the cerebrospinal fluid (Peyron et al. Nat Med 6, 991-997 (2000)). An autoimmune etiology for T1N has been proposed for decades, based on the discovery of a major histocompatibility complex (MHC), human leukocyte antigen (HLA) class II haplotype (DRB1*15:01-DQB1*06:02) association (Matsuki et al. Lancet 339, 1052 (1992)). Up to 98% of narcoleptic patients carry DQB1*06:02 (in strong association with DQA1*01:02 encoding DQ6α/β heterodimers, hereafter referred to as HLA-DQ6), compared to about 25% of normal individuals (Tafti et al. Sleep 37, 19-25 (2014)). Genome-wide association studies (GWAS) in DQ6+ individuals also revealed a risk (odds ratio-1.7) conferred by a single-nucleotide polymorphism (SNP) haplotype, rs1154155-rs1483979; the latter position with G/C alleles is located in the coding region of TRAJ24 gene segment (Hallmayer et al. Nat Genet 41, 708-711 (2009); Han et al. PLoS Genet 9, e1003880 (2013)). Several other GWAS-identified risk SNPs are located within genes that encode proteins functioning in T cell survival and class II antigen presentation, including the purinergic receptor P2YR11, cathepsin H, and the co-stimulatory molecule OX40 ligand (Han et al. PLoS Genet 9, e1003880 (2013); Kornum et al. Nat Genet 43, 66-71 (2011); Faraco et al. PLoS Genet 9, e1003270 (2013)). As CD4⁺ T helper (Tₕ) orchestrate Ab and cytolytic T lymphocyte (CTL) responses, possible contributions from autoantibodies (Cvetkovic-Lopes et al. J Clin Invest 120, 713-719 (2010); Saariaho et al. J Autoimmun 63, 68-75 (2015); Tanaka et al. Sleep 29, 633-638 (2006); Ahmed et al. Sci Transl Med 7, 294ra105 (2015); Luo et al. PLoS One 12, e0187305 (2017)) and CD8⁺ T cells to T1N (using a murine model, Bernard-Valnet et al. Proc Natl Acad Sci U.S.A 113, 10956-10961 (2016) complement the genetic data and a recent study (Latorre et al. Nature 562, 63-68 (2018)) in implicating autoreactive CD4⁺ T cells in the immunopathophysiology of T1N. The risk associated with HLA-DQ6 implies a critical role in pathogenesis for DQ6-restricted CD4⁺ T cells, but the (auto)antigenic specificity of these cells remains unknown.

There remains a need for better methods of diagnosing and treating narcolepsy.

### SUMMARY

Compositions, methods, and kits are provided for diagnosing narcolepsy. Evidence is provided for an autoimmune contribution to the development of narcolepsy, which is associated with loss of neurons supplying the hypocretin (HCRT) neurotransmitter. Susceptibility to type 1 narcolepsy is linked with the HLA-DQ6 allele and a single nucleotide polymorphism (SNP) in the T cell receptor (TCR) gene segment, TRAJ24. In narcolepsy patients, a specific peptide of the secreted form of the HCRT autoantigen is recognized by T cells through antigen presentation restricted to the HLA-DQ allele. It has been discovered that autoreactive T cells specific for the HCRT autoantigen express a set of related TCRs encoded by the TRBV29-1_J2-5 (beta chain) and TRAV6_J24 (alpha chain) genes and carry a SNP allele encoding the leucine variant, TRAJ24L. T cell clonotypes expressing these risk allele-bearing TCRs are expanded in patients who have narcolepsy. It has been found that detection of such autoreactive T cells specific for the HCRT autoantigen is useful in identifying individuals at risk of developing narcolepsy, diagnosing subjects suspected of having narcolepsy, as well as monitoring treatment of patients diagnosed with narcolepsy.

In one aspect, a method of detecting an autoreactive T cell specific for a hypocretin (HCRT) autoantigen is provided, the method comprising: a) obtaining a biological sample comprising T cells from a subject at risk of developing narcolepsy, suspected of having narcolepsy, or diagnosed with narcolepsy; and b) detecting binding of the autoreactive T cell to an antigen presenting cell presenting at its surface the HCRT autoantigen in a complex with human leukocyte antigen (HLA)-DQ6, wherein the autoreactive T cell expresses a T cell receptor that specifically binds to the HCRT autoantigen. In one embodiment, the HLA-DQ6 is encoded by a DQB1*0602 allele.

In certain embodiments, the HCRT autoantigen is a peptide fragment of a prepro-HCRT protein (e.g., prepro-HCRT in its form prior to secretion by neurons) comprising an autoantigen epitope that specifically binds to the HLA-DQ6. Exemplary HCRT autoantigens that can be used in the practice of the methods include HCRT₁₋₁₃ comprising residues 1-13 of prepro-HCRT, HCRT₂₅₋₃₇ comprising residues 25-37 of prepro-HCRT, HCRT₅₆₋₆₉ comprising residues 56-69 of prepro-HCRT, HCRT₈₇₋₁₀₀ comprising residues 87-100 of prepro-HCRT, and HCRT₈₇₋₉₇ comprising residues 87-97 of prepro-HCRT. The foregoing numbering is relative to the human HCRT precursor protein (SEQ ID NO:35), and it is to be understood that the corresponding positions in different HCRT isoforms and HCRT obtained from other species are also intended to be encompassed by the present invention. The HCRT autoantigen peptides may comprise postexpression modifications. In certain embodiments, the HCRT autoantigen peptide comprises a C-terminal modification (e.g., amidation).

In certain embodiments, the HCRT autoantigen comprises an amino acid sequence selected from the group consisting of SEQ ID NOS:31-34, or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity thereto, wherein the HCRT autoantigen comprises an HCRT autoantigen epitope that specifically binds to HLA-DQ6.

In certain embodiments, the biological sample is blood, peripheral blood mononuclear cells, lymphoid tissue, cerebrospinal fluid, or nervous system tissue. In some embodiments, the method further comprises isolating the autoreactive T cells specific for and reactive to the HCRT autoantigen from the biological sample.

In certain embodiments, the antigen presenting cell is a dendritic cell or a macrophage. In other embodiments, the antigen presenting cell is a microglial cell (e.g., in the nervous system). In further embodiments, an artificial antigen presenting cell is used such as, but not limited to, an MHC multimer (i.e., HLA-DQ6 multimer), a cellular artificial antigen presenting cell (e.g., fibroblasts or other cells genetically modified to express HLA-DQ6 and other T cell stimulating proteins), or an acellular antigen presenting cell (e.g., biocompatible particle such as a microparticle or nanoparticle carrying T cell stimulating proteins).

In certain embodiments, the method further comprises determining if the T cell receptor is encoded by a TRBV29-1_J2-5 gene and a TRAV6_J24 gene carrying a risk allele encoding TRAJ24L. For example, a TCR alpha gene and a TCR beta gene of the autoreactive T cell can be sequenced to determine if the T cell receptor is encoded by a TRBV29-1_J2-5 gene and a TRAV6_J24 gene carrying the risk allele encoding TRAJ24L. Alternatively, an immunoassay can be performed with an anti-idiotype antibody that specifically binds to the T cell receptor encoded by the TRBV29-1_J2-5 gene and the TRAV6_J24 gene carrying the risk allele encoding TRAJ24L in order to detect the variant TCR on the surface of the autoreactive T cell.

In certain embodiments, the method further comprises detecting activation of the autoreactive T cell. For example, activation of the autoreactive T cell may be detected by performing a TCR-mediated signaling assay using a luciferase reporter gene system (see Examples), a lymphoproliferation assay, or an assay for intracellular or secreted cytokines. In certain embodiments, the method further comprises detecting activation and/or expansion of autoreactive T cells in vivo.

In certain embodiments, the method further comprises diagnosing the subject with narcolepsy by comparing the number of autoreactive T cells specific for the HCRT autoantigen in the biological sample to a reference value for the number of autoreactive T cells specific for the HCRT autoantigen in a control sample isolated from a healthy donor, wherein the subject is diagnosed as having narcolepsy if the number of the autoreactive T cells specific for the HCRT autoantigen is higher in the biological sample than the reference value.

In another embodiment, a method of diagnosing and treating narcolepsy is provided, the method comprising: a) obtaining a biological sample comprising T cells from a subject at risk of developing narcolepsy, suspected of having narcolepsy, or diagnosed with narcolepsy; b) detecting binding of a T cell to an antigen presenting cell presenting at its surface an HCRT autoantigen in a complex with human leukocyte antigen (HLA)-DQ6, wherein the T cell expresses a T cell receptor that specifically binds to the HCRT autoantigen; c) diagnosing the subject by determining if the T cell receptor is encoded by a TRBV29-1_J2-5 gene and a TRAV6_J24 gene carrying a risk allele encoding TRAJ24L, wherein detection of the TRBV29-1_J2-5 gene and the TRAV6_J24 gene carrying the risk allele encoding TRAJ24L in the T cell indicates that the subject has narcolepsy; and d) treating the subject for narcolepsy if the subject is diagnosed with narcolepsy based on the detection of the TRBV29-1_J2-5 gene and the TRAV6_J24 gene carrying the risk allele encoding TRAJ24L. In some embodiments, the method comprises sequencing the TCR alpha gene and the TCR beta gene of the T cell bound to the antigen presenting cell to determine if the T cell receptor is encoded by a TRBV29-1_J2-5 gene and a TRAV6_J24 gene carrying the risk allele encoding TRAJ24L. In other embodiments, the method comprises performing an immunoassay with an anti-idiotype antibody that specifically binds to the variant T cell receptor encoded by the TRBV29-1_J2-5 gene and the TRAV6_J24 gene carrying the risk allele encoding TRAJ24L.

In certain embodiments, the method further comprises detecting the risk allele encoding TRAJ24L in a T cell that is clonally expanded in vivo in the subject, wherein expansion of T cells having the risk allele encoding TRAJ24L further indicates that the subject has narcolepsy.

Individuals diagnosed with narcolepsy by the methods described herein can be treated in various ways. For example, narcolepsy can be treated with a central nervous system stimulant such as methylphenidate, amphetamine, dextroamphetamine, modafinil, or armodafinil; a norepinephrine reuptake inhibitor (NRI) such as atomoxetine; a selective serotonin reuptake inhibitor or tricyclic antidepressant, such as clomipramine, imipramine, or protriptyline; or sodium oxybate; or a combination thereof.

In another aspect, a method is provided for detecting an autoreactive T cell specific for a hypocretin (HCRT) autoantigen, the method comprising: a) obtaining a biological sample comprising T cells from a subject; b) eliciting an antigen-specific T cell response by contacting the T cells with an antigen presenting cell presenting at its surface the HCRT autoantigen in a complex with HLA-DQ6; and c) detecting the antigen-specific T cell response.

In another aspect, a method is provided for diagnosing and treating narcolepsy, the method comprising: a) obtaining a biological sample comprising T cells from a subject at risk of developing narcolepsy, suspected of having narcolepsy, or diagnosed with narcolepsy; b) detecting binding of an autoreactive T cell to an antigen presenting cell presenting at its surface the HCRT autoantigen in a complex with human leukocyte antigen (HLA)-DQ6; c) diagnosing the subject with narcolepsy by comparing the number of autoreactive T cells specific for the HCRT autoantigen in the biological sample to a reference value for the number of autoreactive T cells specific for the HCRT autoantigen in a control sample isolated from a healthy donor, wherein the subject is diagnosed as having narcolepsy if the number of the autoreactive T cells specific for the HCRT autoantigen is higher in the biological sample than the reference value; and d) treating the subject for narcolepsy if the subject is diagnosed with narcolepsy based on the number of the autoreactive T cells specific for the HCRT autoantigen in the biological sample. Treatment of narcolepsy may comprise, for example, administering a central nervous system stimulant such as methylphenidate, amphetamine, dextroamphetamine, modafinil, or armodafinil; a norepinephrine reuptake inhibitor (NRI) such as atomoxetine; a selective serotonin reuptake inhibitor or tricyclic antidepressant, such as clomipramine, imipramine, or protriptyline; or sodium oxybate; or a combination thereof.

In another aspect, an isolated HCRT autoantigenic peptide is provided comprising an amino acid sequence selected from the group consisting of SEQ ID NOS:31-34, or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% sequence identity thereto, wherein the immunogenic peptide comprises an HCRT autoantigen T cell epitope.

In some embodiments a kit is provided for practicing the methods described herein, where the kit contains reagents suitable for detection of autoreactive T cells specific for an HCRT autoantigen. Such kits may include, for example, a detectably labeled HCRT autoantigen peptide and antigen presenting cells for detecting autoreactive T cells. The antigen presenting cell included in the kit may be a dendritic cell, macrophage, or microglial cell, or an artificial antigen presenting cell (e.g., MHC multimer (i.e., HLA-DQ6 multimer), a cellular artificial antigen presenting cell, or an acellular antigen presenting cell). Kits may further comprise reagents for performing an immunoassay or functional assay to detect activation of the autoreactive T cells, buffers, and the like for use in detecting autoreactive T cells specific for an HCRT autoantigen; and instructions for use. The kit reagents may be provided as an in vitro diagnostic (IVD) device.

In certain embodiments, the kit comprises an HCRT autoantigen peptide comprising an amino acid sequence selected from the group consisting of SEQ ID NOS:31-34, or a sequence displaying at least about 70-100% sequence identity thereto, including any percent identity within this range, such as 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% sequence identity thereto, wherein the HCRT autoantigen peptide comprises an HCRT autoantigen T cell epitope.

In another aspect, an isolated antigen presenting cell is provided presenting at its surface an HCRT autoantigen peptide, described herein, in a complex with human leukocyte antigen (HLA)-DQ6. In certain embodiments, a composition is provided comprising the antigen presenting cell and a pharmaceutically acceptable excipient.

In another aspect, an isolated autoreactive T cell expressing a T cell receptor encoded by a TRBV29-1_J2-5 gene and a TRAV6_J24 gene carrying a risk allele encoding TRAJ24L is provided, wherein the T cell receptor forms a complex with an antigen presenting cell presenting at its surface an HCRT autoantigen peptide, described herein, in a complex with human leukocyte antigen (HLA)-DQ6.

In another aspect, an isolated regulatory T cell expressing a T cell receptor encoded by a TRBV29-1_J2-5 gene and a TRAV6_J24 gene carrying a risk allele encoding TRAJ24L is provided, wherein the T cell receptor forms a complex with an antigen presenting cell presenting at its surface an HCRT autoantigen peptide, described herein, in a complex with human leukocyte antigen (HLA)-DQ6. In another embodiment, a composition is provided comprising the regulatory T cell expressing a T cell receptor encoded by a TRBV29-1_J2-5 gene and a TRAV6_J24 gene carrying a risk allele encoding TRAJ24L, described herein. In certain embodiments, the composition further comprises a pharmaceutically acceptable excipient.

In another embodiment, a composition is provided comprising regulatory T cell expressing a T cell receptor encoded by a TRBV29-1_J2-5 gene and a TRAV6_J24 gene carrying a risk allele encoding TRAJ24L complexed with an antigen presenting cell presenting at its surface an HCRT autoantigen peptide, described herein, in a complex with human leukocyte antigen (HLA)-DQ6. In another embodiment, the composition further comprises a pharmaceutically acceptable excipient.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1 shows a schematic illustration of the approach. (A) An array of overlapping peptides covering the entire candidate autoantigen HCRT is tested for HLA-DQ6 binding. (B) DQ6-HCRT_{peptide} tetramers are synthesized based on the DQ6-binding cores determined in (A). (C) CD4⁺ T cells are isolated from PBMCs of DQ6⁺ patients and controls using negative selection by magnetic activated cell sorting (MACS); purified CD4⁺ T cells are co-stained with candidate tetramers and antibodies distinguishing cell types (i.e., anti-CD4, anti-CD19). DQ6-HCRT_{peptide} tetramer⁺/CD4⁺ cells are sorted by single cell index sorting (iFACS). The iFACS-sorted cells likely include tetramer⁺ clones with various tetramer binding ranks and clone size, including either T1N-relevant (red clones expressing public/related TCRs or orange clones expressing private TCRs) or irrelevant (green clones). Unexpanded clones expressing TCR risk alleles may also be observed in DQ6⁺ controls, as DQ6-restricted selection may occur similarly in patients and controls and T1N development is thought to rely largely on antigen-driven clonal expansion. (D) Deep sequencing of TCR and phenotypic transcripts in sorted single cells allows further assessment of T1N-associated gene signatures of tetramer⁺ clones including both in vivo clonal expansion and expression of the TCR risk allele. (E-H) The DQ6-restricted TCR sequences are validated for ability to generate an expressed α/β TCR that binds relevant tetramers and signals after stimulation with relevant peptide epitopes.

FIGS. 2A-2E show DQ6-binding cores in prepro-HCRT and their structural impacts. (FIG. 2A) 30 overlapping peptides covering prepro-HCRT were added, individually, into a reaction containing soluble DQ6, bio-EBV₄₉₀₋₅₀₃ peptide, and HLA-DM (a peptide loading catalyst). DQ6-associated bio-EBV₄₉₀₋₅₀₃ was measured at steady state by ELISA⁴⁴. % Competition=1-% DQ6/EBV binding. Strong (>75% Competition, in colors) and weak (50-75% Competition, in gray) DQ6 binders with predicted cores (bolded) are aligned. (FIG. 2B) Top view of HCRT₅₆₋₆₉ (orange) in the peptide-binding groove of DQ6 (α/β, light green/blue surface, PDB: 6DIG). Core residues are indicated. (FIG. 2C) Alignment of DQ6-HCRT₅₆₋₆₉ (α/β, light green/blue cartoon; peptide, orange stick) and DQ6-HCRT₁₋₁₃ structures²⁷ (α/β, dark green/blue; peptide, magenta) illustrating three regions in DQ6 with noticeable conformation differences (detailed in FIG. 8E). (FIG. 2D) Side view of DQ6 (p chain removed to reveal peptides) in complex with HCRT₁₋₁₃ (PDB: 1UVQ) and DQ6-HCRT₅₆₋₆₉ (PDB: 6DIG) and models of HCRT₂₅₋₃₇ and HCRT₈₇₋₁₀₀ (sticks in the same color as in FIG. 2A). Arrow indicates predicted positioning for interaction of TCRα/βCDR3s. (FIG. 2E) Zoom-in of the 9-aa core registers of peptides shown in (FIG. 2D). Arrows indicate TCR (up) or DQ6 (down) facing resides.

FIGS. 3A-3E show tetramer+/CD4⁺ T cells in DQ6⁺ donors. (FIG. 3A) Dot-plots of CD4⁺ T cells stimulated in vitro with the indicated peptides followed by staining with the indicated tetramers. Frequencies (%) of tetramer+/CD4⁺ T cells are indicated. (FIG. 3B) Comparison between frequencies of tetramer⁺ T cells from the same T1N donor (paired as indicated) with and without peptide stimulation. Significance (P<0.05, bolded) is determined using the paired samples t-test (n=13 donors). (FIG. 3C) Enrichment of DQ6-HCRT₅₆₋₆₉ tetramer+/CD4⁺ T cells in HCRT₈₇₋₁₀₀ peptide stimulated culture. (FIG. 3D) Frequencies or (FIG. 3E) tetramer binding ranks (mean index fluorescence intensity) of tetramer⁺ T cells from patients and controls stained with the indicated tetramers. Significance (P<0.05, bolded) is determined using the unequal variances t-test (sample information summarized in FIG. 4A.

FIGS. 4A-4C show sequence analysis of tetramer+/CD4⁺ cells from DQ6⁺ donors. (FIG. 4A) Summary of single-cell analysis. Subject numbers indicate the order of pairs (Control/Patient) used in blinded experiments. “x” and “#” indicate vaccinations using Pandemrix or seasonal trivalent inactivated influenza vaccine, including pandemic H1N1 (TIV), respectively. BD: blood draw. Cells represent fully identified TCRα/β gene information and productive CDR3 sequences. Clonotypes represent unique, paired α/β CDR3s. Identical TCR clonotypes identified by different tetramers are aligned, but the alignment across subjects does not reflect related TCRs. Highly expanded clonotypes (>5 total isolates per subject) are shaded. (FIG. 4B) Percent of cells with productive α/β CDR3 sequences among all wells that yielded TCR sequences with the indicated tetramer specificity or (FIG. 4C) frequency of tetramer⁺ cells that are expanded in vivo within each subject with the indicated tetramer specificity are analyzed as control/patient pairs that were stained/sorted in the same experiment. Significance (P<0.05, bolded) between patients and controls within each tetramer category is determined using the paired samples t-test. Unequal variances t-test is used for comparison between two specificity categories. ns: P>0.05.

FIGS. 5A-5E show a T1N-associated signature in the expanded TRAJ24L clonotypes. (FIG. 5A) A heat map illustrating 9 groups (1-IX) of cells from multiple subjects sharing identical paired TRBJ/TRAJ genes. Number of isolates of the same clone is proportional to the color intensity. (FIG. 5B) Frequencies of V or J genes used in multi-hit TCRα/β clonotypes compared with all sequenced cells. Arrows indicate genes used in TRBV29-1_J2-5/TRAV6_J24-expressing cells, with fold increase compared to “All Cells”. (FIG. 5C) Summary of tetramer-identified clones expressing TRBV29-1_J2-5/TRAV6_J24. The L variant encoded by the risk SNP allele is indicated in red. The 3 eTRAJ24L clonotypes are shaded. (FIG. 5D) GLIPH analysis of CDR3P motifs. Each dot represents one motif: global (red), local (blue), single (gray). The Radius of dots is proportional to the final score. (FIG. 5E) Tetramer binding rank of individual eTRAJ24L⁺ cells. Bar graphs represent the MFI with SEM of tetramer-associated signal on the surface of CD4⁺/tetramer⁺ single cells from the indicated subject-tetramer category. Each open symbol represents the index tetramer Fl of one eTRAJ24L+ cell identified from the same subject-tetramer category as the corresponding bar graph.

FIGS. 6A-6F show validation of tetramer binding and TCR signaling. (FIG. 6A) TCRs tested by transfection into Jurkat-reporter system²⁰. (FIG. 6B) Dot-plots of Jurkat transfectants expressing TCRs as shown in (FIG. 6A). Expression of GFP indicates a successful transfection of plasmids directing the expression of TCRs (see methods). (FIG. 6C) Staining of TCR⁺ Jurkat cells with the indicated DQ6-HCRT_{peptide} tetramer. Percentages of tetramer⁺ cells are indicated. (FIG. 6D) MFIs of the tetramer-associated signal on the surface of TCR⁺ cells are compared. (FIG. 6E) Jurkat cells expressing the indicated TCR were incubated with DQ6-expressing APCs (HLA class II⁻ K562 cells transfected with DQ6) in the presence of HCRT peptides or control stimuli: PBS (negative) or anti-CD3/anti-CD28 Abs (positive). Luciferase activity reflecting TCR-mediated signaling was measured and analyzed. (FIG. 6F) An independent TCR-27 Jurkat-transfectant was tested in response of HCRT₈₇₋₉₇-NH2 compared to HCRT₈₇₋₁₀₀ using a similar luciferase activity assay and the responses were time-dependent. All error bars represent SEM. Significance (P<0.05, bolded) is determined using t-test; n=3 in (FIGS. 6D and 6F), n=5 in (FIG. 6E).

FIG. 7A-7D show a phenotypic analysis of the DQ6-HCRT_{peptide} tetramer⁺/CD4⁺ cells from DQ6⁺ donors. (FIG. 7A) Sequenced cells from controls (FIG. 7C) or patients (P) in each set (FIG. 4A) are clustered as indicated. Each color in the horizontal bar above the heat map represents one donor. Red indicates the presence of mRNA in the cell (genes indicated to the left of the heat map). *Different sets of primers were used to amplify IFN-γ mRNAs in cells from Set A and Set B donors. (FIG. 7B) Frequencies of expanded versus unexpanded cells expressing the indicated transcript. (FIG. 7C) Phenotypes of expanded or unexpanded TRAJ24L⁺ clones versus TRAJ24F⁺ clones. Each color in the horizontal bar represents one subject-tetramer category of single cells. Patients or controls are clustered as indicated under expanded or unexpanded groups. The expanded TRAJ24L cluster includes the unexpanded clonotype from P3, as it only differs from other clonotypes by 1 residue in both α/β CDR3 sequences. (FIG. 7D) Frequencies of cells from the indicated cluster expressing the indicated transcript in (FIG. 7C). The scale of color intensity is the same as in (FIG. 7B).

FIGS. 8A-8E show NetMHCIIpan3.2 prediction and structural analysis of HCRT binding to DQ6. (FIG. 8A) Predicted binding to DQ6 of the 30 overlapping 15-mer peptides used in the peptide loading assay (FIG. 2A). The % Rank reflects how the predicted affinity for a given peptide ranks compared to a set of 200,000 random natural peptides of the same length²⁶. The value (100-% Rank) is positively correlated to the predicted binding affinity. Strong binders ranking in the top 2% are highlighted using a similar color scheme as shown in FIG. 2A. Gray indicates a predicted strong binder that was not determined by the empirical experiment. HCRT₁₁₅ (in magenta) was predicted as a weak binder. (FIG. 8B) A comparison between NetMHCIIpan3.2 prediction and the empirical result (FIG. 2A). Prediction showed 100% sensitivity and specificity (predicted all 9 binders as shown in FIG. 2A) when 85% was used as a prediction cutoff and 75% competition was used as the empirical cutoff for DQ6 binding. (FIG. 8C and FIG. 8D) Predicted binding of all possible (FIG. 8C) 15-mer or (FIG. 8D) 9-mer peptides derived from prepro-HCRT to DQ6. Strong binders as shown were predicted using different peptide-length parameters in NetMHCIIpan3.2 and 9-aa binding cores were bolded. Colors match the color scheme in (FIG. 8A). Gray indicates predicted strong binding cores that are outside of or overlap with the region with strong binders determined by the empirical experiment. Notably, the software failed to predict the X-ray structure-determined core²⁷, LPSTKVSWA in HCRT₁₋₁₅ (in blue). (FIG. 8E) Zoom-in of the three regions (FIG. 8E(a)-8E(c)) shown in FIG. 2C. FIG. 8E(a) The polar P1N of HCRT₅₆₋₆₉ forms three hydrogen (H)-bonds (dotted lines, not in DQ6-HCRT₁₋₁₃) causing side chain rotations (arrows) at H81p and N82P. FIG. 8E(b) P3A and P4A of HCRT₅₆₋₆₉ abolished three H-bonds observed in DQ6-HCRT₁₋₁₃ allowing DQ6α1 helix to move inward with side chain rotations at Q60α and R64α. FIG. 8E(c) The hydrophilic P7L disabled two H-bonds observed in DQ6-HCRT₁₋₁₃ forcing DQ6P1 helix to move outward as reflected by side chain rotations at Y60β, Q64β and E66β.

FIGS. 9A-9E show in vitro enrichment of DQ6-EBV₄₈₇₋₄₉₉ tetramer⁺/CD4⁺ cells. (FIG. 9A) Dot-plots showing time dependent enrichment of DQ6-EBV₄₈₇₋₄₉₉ tetramer⁺/CD4⁺ T cells after in vitro stimulation with the EBV₄₈₆₋₅₀₀ peptide (see methods). No enrichment was observed for cells binding the control DQ6-HCRT₁₋₁₃ tetramer or after culture without the EBV peptide. Frequencies (%) of tetramer+/CD4⁺ cells are indicated. (FIGS. 9B-9D) In vitro stimulation of cells from other donors suggests that our approach enriches tetramer⁺ T cells from most, but not all donor samples. (FIG. 9E) A comparison of time dependent enrichment of DQ6-EBV₄₈₇₋₄₉₉ tetramer⁺/CD4⁺ T cells in the four donors. All donors used in the EBV stimulation experiments are narcoleptic patients with unknown EBV infection history.

FIGS. 10A-10D show tetramer binding rank of cells that are expanded in vivo for the DQ6-HCRT₁₋₁₃ tetramer (FIG. 10A), DQ6-HCRT₂₅₋₃₇ tetramer (FIG. 10B), DQ6-HCRT₅₆₋₆₉ tetramer (FIG. 10C), and DQ6-HCRT₈₇₋₁₀₀ tetramer (FIG. 10D). Bar graphs with standard error of mean (SEM) represent the MFI of tetramer-associated signal on sorted tetramer+/CD4⁺ single cells from the indicated subject (FIG. 4A). Each open symbol represents the index tetramer Fl of one expanded cell identified from the indicated subject. Control: dark gray, patient: light gray. All values are normalized with background MFI of CD4⁺ population.

FIG. 11 shows isolation of DQ6-HCRT_{peptide} tetramer⁺ cells using iFACS. Gating strategy for iFACS. 3-5 million MACS-isolated CD4⁺ T cells per subject sample were tetramer-stained and screened by iFACS to sort a 96-well plate of single tetramer⁺ cells. Singlets, live cells, and CD19⁻/CD4⁺/tetramer⁺ cells were gated as indicated.

FIGS. 12A-12F show evaluation of iNKT-like TCRs. (FIG. 12A) Comparable numbers and frequencies of iNKT-like TCRs found in two datasets obtained by single CD4⁺ T cell sorting and TCR sequencing: one described in this manuscript and one in which T cells activated by M. tuberculosis peptides were isolated²⁰. (FIG. 12B) Nucleofection of Jurkat cells using a mixture of plasmids, each directing the expression of one TCR in (a), was used to construct a library of iNKT-like TCR transfectants. Tetramer staining of the resultant transfectant library was analyzed as in FIG. 6D. (FIG. 12C) A single clone expressing an iNKT-like TCR was FACS-sorted from the library and expanded. The dot-plot shows the expression of the iNKT-like TCR in this clonal line. (FIGS. 12D and 12E) Tetramer staining of the clonal line was analyzed as in FIGS. 6C and 6D. (FIG. 12F) HCRT epitopes were unable to trigger signaling in these iNKT-like TCR transfectants, as indicated by the background level of luciferase activity.

FIGS. 13A and 13B show a phenotypic analysis. (FIG. 13A) Frequencies of sequenced cells from controls (C) or patients (P) or altogether (A) expressing the indicated transcript clustered as indicated. (FIG. 13B) Frequencies of cells from each subject-tetramer category expressing the indicated transcript (color-coded) were compared with the average (set to zero) for the same transcript. TBET (TBX21), PRF1, and IFN-γ share very similar patterns. The detection frequencies of GATA3, TNF-α, TGF-β, and RUNX1 in all categories are around the average level.

FIGS. 14A and 14B show expression of (FIG. 14A) CD25 and (FIG. 14B) CD127 on the surface of sequenced cells from Set A donors. Bar graphs represent the MFI with SEM of Ab staining signal on the surface of sequenced single cells (FIG. 4A) in the indicated subject-tetramer category. Each open symbol represents the index Ab Fl of one expanded cell identified from the same subject-tetramer category as the corresponding bar graph. Control: blue, patient: red. All staining is at the background level.

### DETAILED DESCRIPTION OF EMBODIMENTS

Compositions, methods, and kits are provided for diagnosing and treating narcolepsy. Susceptibility to type 1 narcolepsy is linked with the human leukocyte antigen (HLA)-DQ6 allele and a single nucleotide polymorphism (SNP) in the T cell receptor gene segment TRAJ24. The presence of T cells reactive with hypocretin (HCRT) autoantigens is also associated with development of narcolepsy. Compositions and methods are provided for detecting autoreactive T cells specific for hypocretin (HCRT) autoantigens presented by HLA-DQ6. Additionally, a method of treating a subject for narcolepsy is provided comprising administering a therapeutically effective amount of a composition comprising T regulatory cells expressing a variant T cell receptor encoded by the TRBV29-1_J2-5 and TRAV6_J24 genes to the subject.

Before the present compositions, methods, and kits are described, it is to be understood that this invention is not limited to the particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Biological sample. The term “sample” with respect to an individual encompasses any sample comprising T cells such as blood and other liquid samples of biological origin, solid tissue samples such as a biopsy specimen or cancerous tissue from a surgically resected tumor, malignant effusion fluid samples, or tissue cultures or cells derived or isolated therefrom, and the progeny thereof. The definition also includes samples that have been manipulated in any way after their procurement, such as by treatment with reagents; washed; or enrichment for certain cell populations, such as T cells. The definition also includes samples that have been enriched for particular types of molecules, e.g., nucleic acids, polypeptides, etc.

The term “biological sample” encompasses a clinical sample. The types of “biological samples” include but are not limited to: tissue obtained by surgical resection, tissue obtained by biopsy, cells in culture, cell supernatants, cell lysates, tissue samples, organs, bone marrow, blood, plasma, serum, cerebral spinal fluid, fine needle aspirate, lymph node aspirate, cystic aspirate, a paracentesis sample, a thoracentesis sample, and the like.

The terms “obtained” or “obtaining” as used herein can also include the physical extraction or isolation of a biological sample (e.g., comprising T cells) from a subject. Accordingly, a biological sample can be isolated from a subject (and thus “obtained”) by the same person or same entity that subsequently performs diagnostic assays on the T cells in the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

The terms “peptide” and “protein” refer to a polymer of amino acid residues and are not limited to a minimum length. Thus, polypeptides, peptides, oligopeptides, dimers, multimers, and the like, are included within the definition. Both full length proteins and fragments thereof are encompassed by the definition. The terms also include postexpression modifications of the protein, for example, amidation, glycosylation, acetylation, phosphorylation, hydroxylation, oxidation, and the like.

By “immunogenic fragment” is meant a fragment of an immunogen which includes one or more epitopes that can stimulate an immune response, including an antigen-specific T cell response. Immunogenic peptides will typically range between 2 to 15 amino acids in length, including any length within this range such as 2, 3, 4, 5, 6, 7, 8, 9, 10, 11, 12, 13, 14, or 15 amino acids in length. In some embodiments, the immunogenic peptide is at least 2, at least 3, at least 5, at least 7, at least 9, at least 10, at least 11, or at least 12 amino acids in length.

As used herein, the term “epitope” generally refers to the site on an antigen which is recognized by a T-cell receptor (e.g., on a T cell) and/or an antibody. The epitope may be contained in a short peptide derived from a protein antigen or part of a protein antigen. Several different epitopes may be carried by a single antigenic molecule. The term “epitope” may also include modified amino acids. Epitopic determinants usually consist of chemically active surface groupings of molecules such as amino acids or sugar side chains and usually have specific three-dimensional structural characteristics, as well as specific charge characteristics.

An immunogenic fragment can be generated from knowledge of the amino acid and corresponding DNA sequences of an antigen (e.g., HCRT autoantigen), as well as from the nature of particular amino acids (e.g., size, charge, etc.) and the codon dictionary, without undue experimentation. See, e.g., Ivan Roitt, Essential Immunoloqy, 1988; Kendrew, supra; Janis Kuby, Immunology, 1992 e.g., pp. 79-81. Some guidelines in determining whether a protein will stimulate a response, include: Peptide length-typically the peptide is about 8 or 9 amino acids long to fit into a MHC class I complex and about 13-25 amino acids long to fit into a class II MHC complex. Peptides may be longer than these lengths. For example, a longer peptide may be needed if it is partially degraded in cells. The peptide may contain an appropriate anchor motif which will enable it to bind to various class I or class II molecules with high enough specificity to generate an immune response (See Bocchia, M. et al, Specific Binding of Leukemia Oncogene Fusion Protein Pentides to HLA Class I Molecules, Blood 85:2680-2684; Englehard, V H, Structure of peptides associated with class I and class II MHC molecules Ann. Rev. Immunol. 12:181 (1994)).

The terms “immunogenic” protein or peptide refer to an antigen having an amino acid sequence which elicits an immunological response, including an antigen-specific T cell response. An “immunogenic” protein or peptide, as used herein, includes the full-length sequence of the protein in question, including the precursor and mature forms, analogs thereof, or immunogenic fragments thereof.

As used herein, the term “T cell epitope” refers generally to those features of a peptide structure which are capable of inducing a T cell response.

An “immunological response” to an antigen or composition is the development in a subject of a humoral and/or a cellular immune response to an antigen present in the composition of interest. For purposes of the present invention, a “humoral immune response” refers to an immune response mediated by antibody molecules, while a “cellular immune response” is one mediated by T-lymphocytes and/or other white blood cells. One important aspect of cellular immunity involves an antigen-specific response by cytotoxic T cells (CTLs). CTLs have specificity for peptide antigens that are presented in association with proteins encoded by the major histocompatibility complex (MHC) and expressed on the surfaces of cells. CTLs help induce and promote the destruction or lysis of cancerous cells, infected cells, or damaged cells. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4⁺ and CD8⁺ T-cells.

The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, e.g., Erickson et al., J. Immunol. (1993) 151:4189-4199; Doe et al., Eur. J. Immunol. (1994) 24:2369-2376.

Methods of measuring a cell-mediated immune response include measurement of intracellular cytokines or cytokine secretion by T-cell populations, or measurement of epitope specific T-cells (e.g., by the tetramer technique) (reviewed by Malyguine et al. (2012) Cells 1(2):111-126, Shafer-Weaver et al. (2003) J. Transl. Med. 1(1):14, Takagi et al. (2017) Biochem. Biophys. Res. Commun. 492(1):27-32, Jerome et al. (2003) Apoptosis 8(6):563-571, Hermans et al. (2004) J. Immunol. Methods 1; 285(1):25-40, van Baalen et al. (2008) Cytometry A 73(11):1058-1065, McMichael and O'Callaghan (1998) J. Exp. Med. 187(9)1367-1371, Mcheyzer-Williams et al. (1996) Immunol. Rev. 150:5-21, Lalvani et al. (1997) J. Exp. Med. 186:859-865; herein incorporated by reference.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer) as well as those in which prevention is desired (e.g., those with increased susceptibility to cancer, those suspected of having cancer, etc.).

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Pharmaceutically acceptable excipient or carrier” refers to an excipient that may optionally be included in the compositions of the invention and that causes no significant adverse toxicological effects to the patient. For example, compositions may include one or more “pharmaceutically acceptable excipients or vehicles” such as water, saline, glycerol, polyethyleneglycol, hyaluronic acid, ethanol, etc. Additionally, auxiliary substances, such as wetting or emulsifying agents, pH buffering substances, surfactants and the like, may be present in such vehicles. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of proteins or nucleic acids or other components of a preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in a composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

It will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Diagnostic Gene Signatures of T1N-Related CD4⁺ T Cells

Here, we investigated the molecular linkage between HCRT precursor (prepro-HCRT) and DQ6-restricted T cells, and report the identification of prepro-HCRT-derived peptides that bind DQ6 and the identification of TRAJ24 risk allele-bearing CD4⁺ T cell clonotypes that bind these peptides. Key features distinguishing clonotypes found in T1N patients include the expression of restrictedly paired TCRα/β chains that both are public/semi-public, the TCR-mediated signaling after binding to the C-terminal end of a physiologically processed HCRT neurotransmitter, the in vivo clonal expansion, and the transcription of type 1 T helper (Tₕ1) phenotypic genes. Therefore, our findings fill a critical gap in the current understanding of narcolepsy-related autoimmunity and shed light on disease diagnosis and treatment.

### Results

### Strategy of Approach

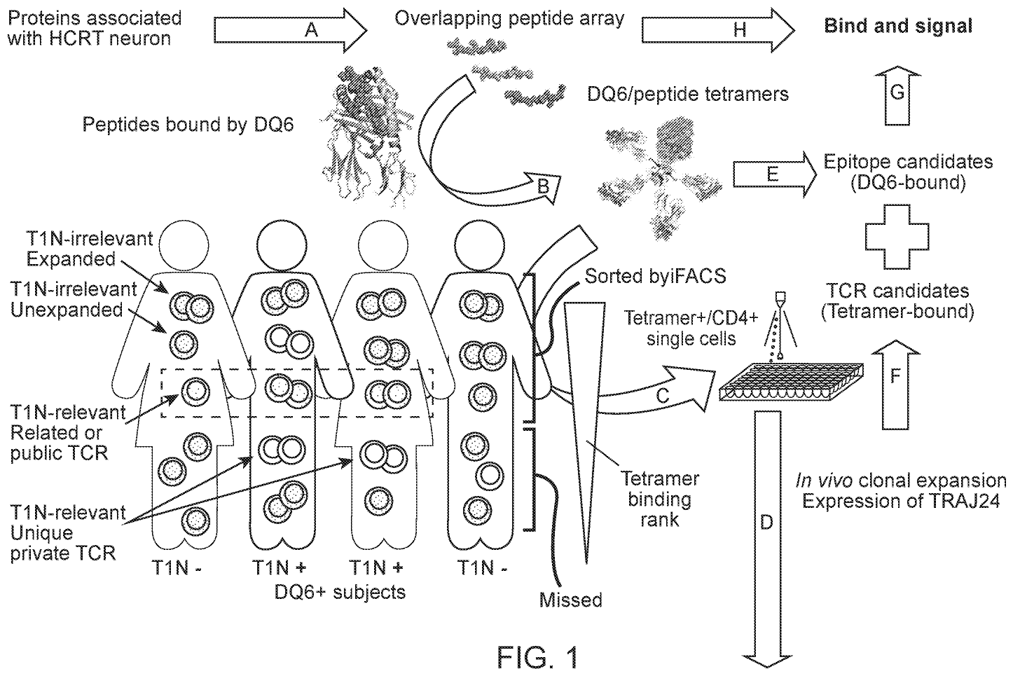

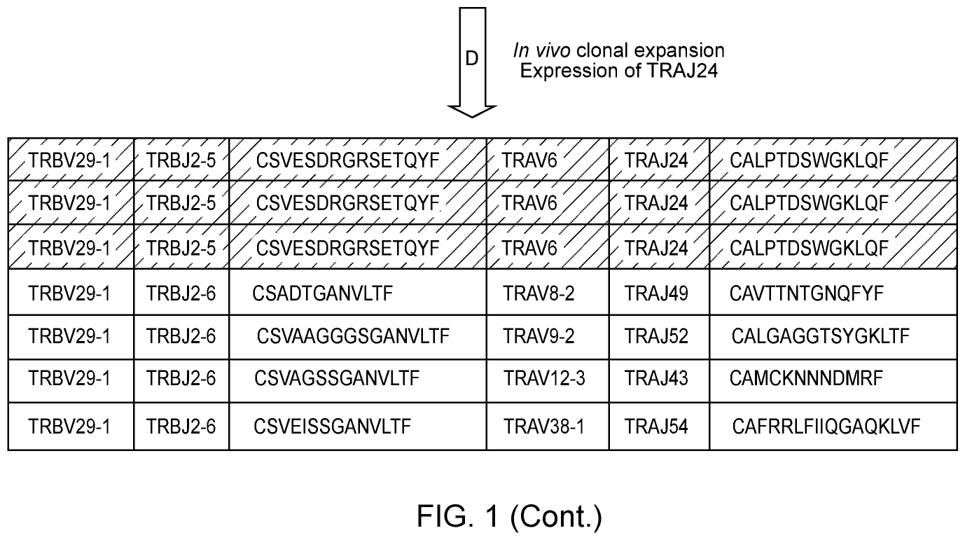

HCRT is the only protein known to be unique to neurons lost in T1N² and thus is a strong candidate autoantigen. To test this possibility, we took an integrated approach of identifying DQ6-binding HCRT peptides and using DQ6-HCRT_{peptide} tetramers to isolate candidate disease-related CD4 T cells (FIG. 1). Importantly, the strategy based on tetramer binding provides information about in vivo clone size and avoids phenotypic alterations caused by in vitro stimulation¹⁶⁻¹⁸. We adapted a well-established pipeline¹⁹⁻²⁰ to deep sequence DQ6-HCRT_{peptide} tetramer⁺/CD4⁺ single cells from DQ6⁺ individuals with/without T1N symptoms. We then examined TCRα/β that share identical/conserved (public/semi-public) α/β complementary-determining region 3 (CDR3) sequences to identify in vivo clonal expansions. Among these, we focused on those that express a GWAS-identified risk SNP allele in the TRAJ24 gene, as potential T1N-associated autoreactive T cells. Previous work shows that tetramer staining does not ensure TCR signaling of pathogen-specific T cells²⁰⁻²² or tumor infiltrating CTLs²³⁻²⁴ in response to HLA-peptide complexes on antigen presenting cells (APCs). In light of this, tetramer binding likely also overestimates the truly reactive (bind and signal) T cell population in the autoimmune context. We therefore tested candidate TCRα/β in a TCR-deficient Jurkat-reporter system²⁰ to prove their capacity to mediate DQ6-HCRT_{peptide} _{^{−}} triggered TCR signaling. The single-cell sequencing pipeline¹⁹ also allowed linking phenotypic features with candidate TCRα/β from each individual cell.

### Epitopes and Binding Registers in HCRT

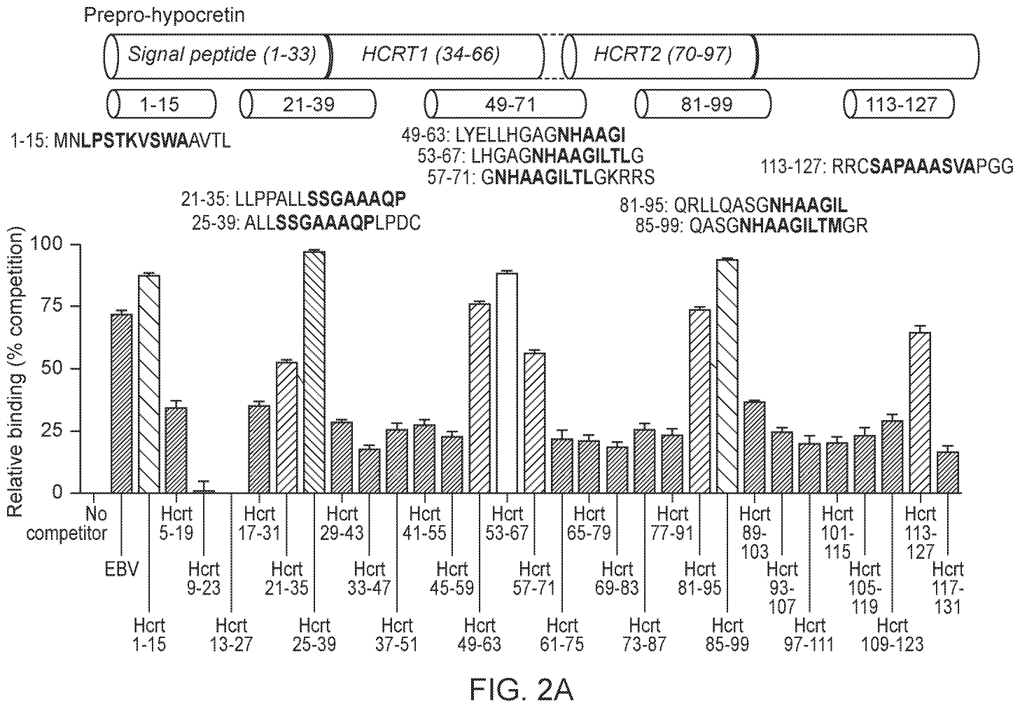

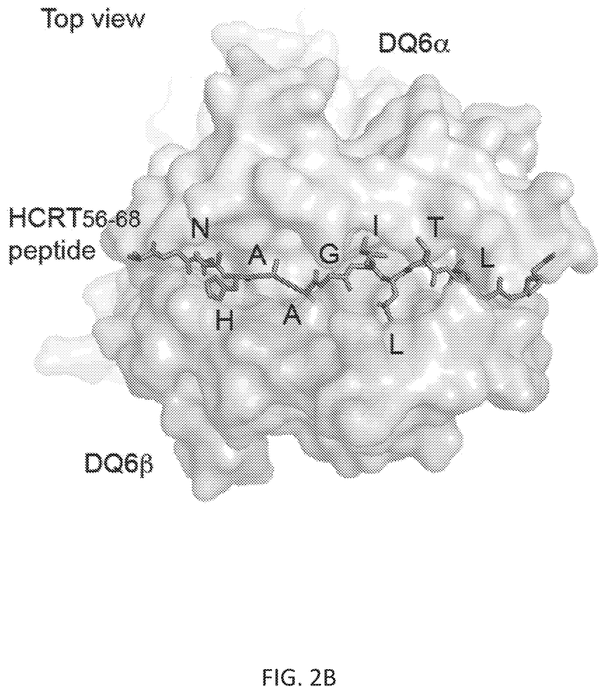

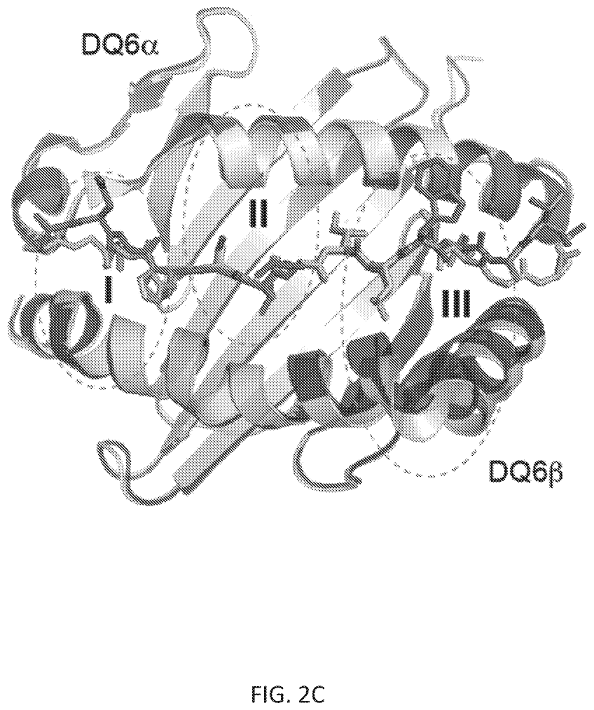

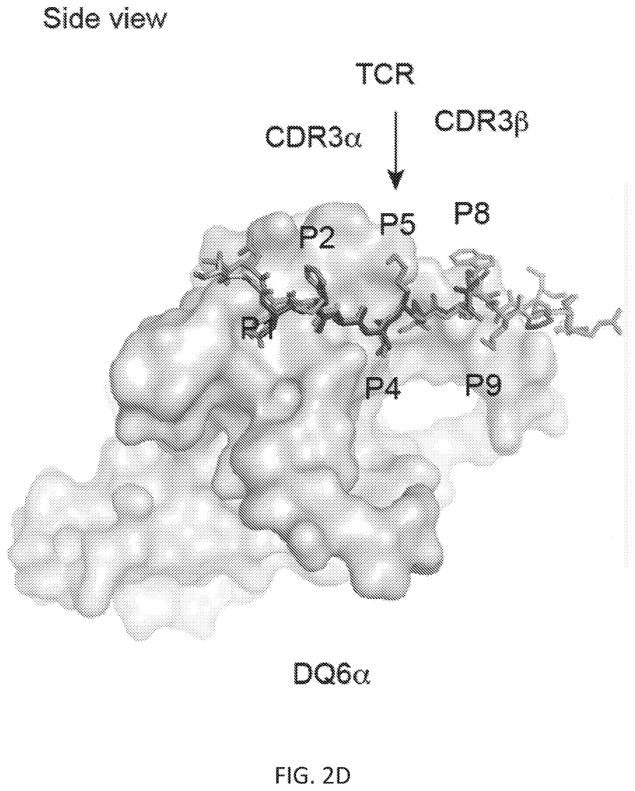

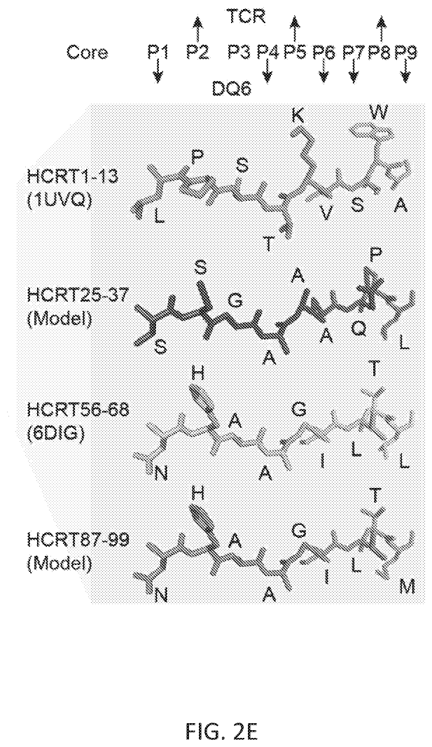

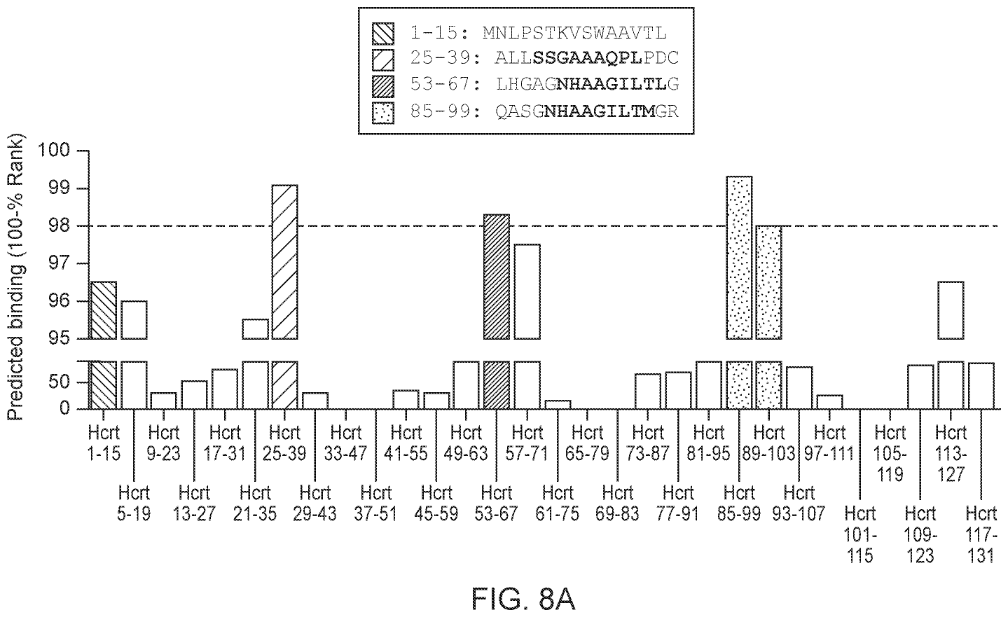

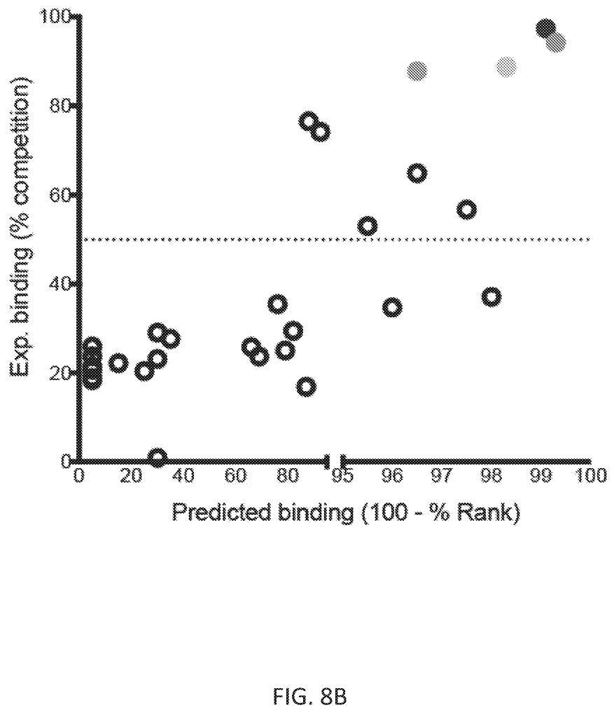

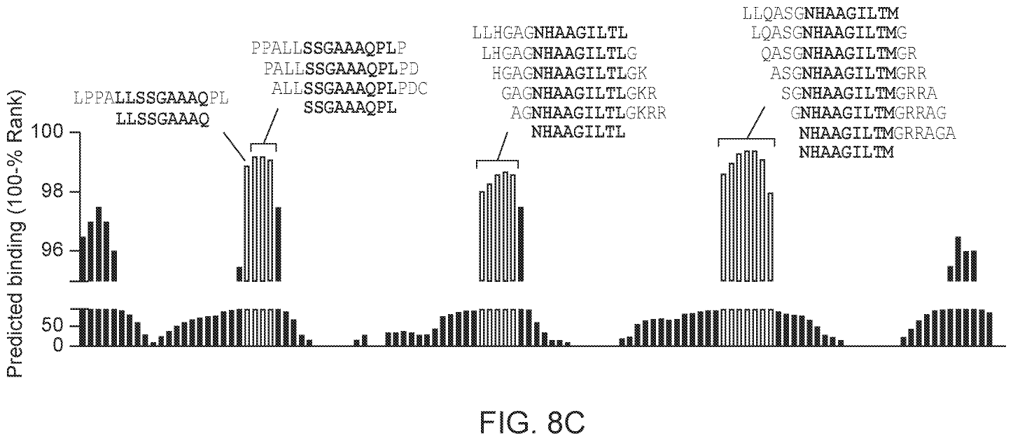

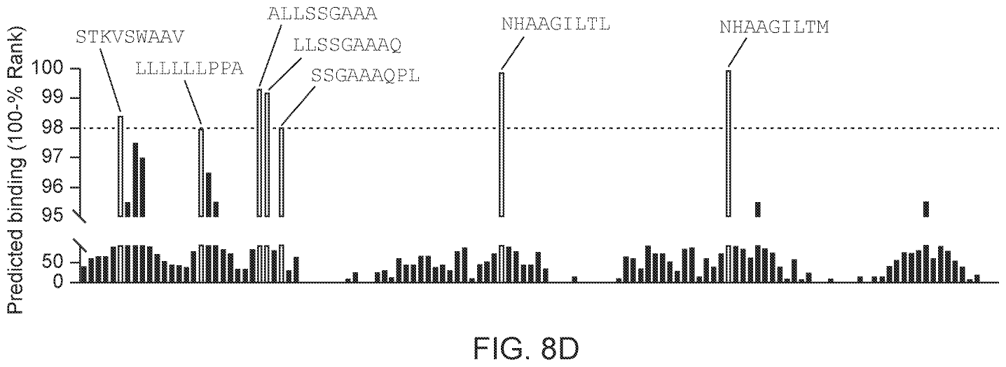

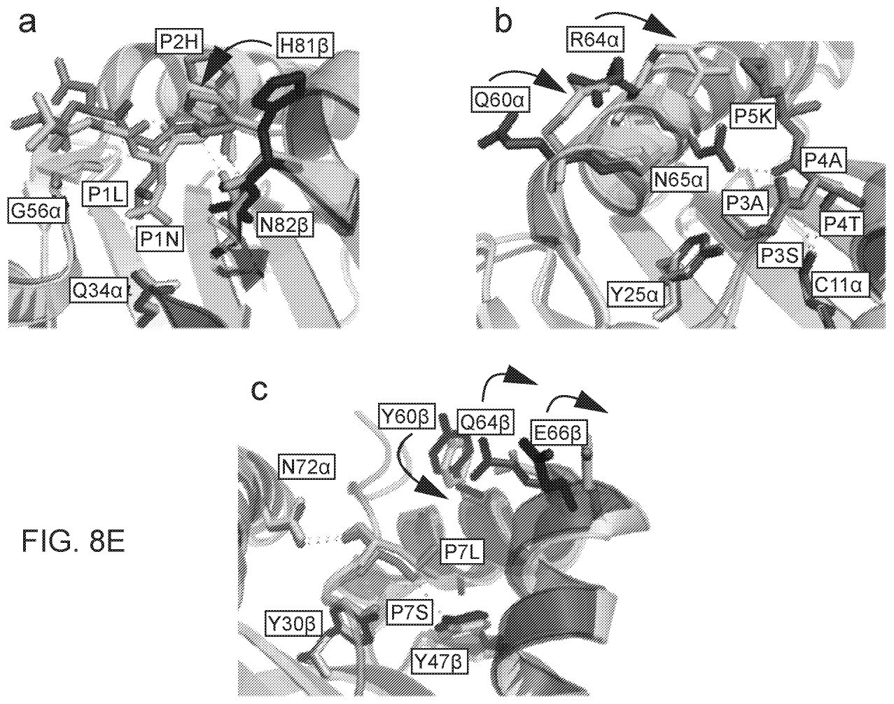

As any peptide derived from prepro-HCRT may be presented by DQ6, we tested overlapping 15-mer peptides, offset by 4 amino acids (aa), covering the entire prepro-HCRT sequence (Table 1). We used a competition assay in which each peptide was examined for the ability to inhibit DQ6-binding of EBV₄₈₆₋₅₀₀, a known epitope derived from Epstein-Bar virus²⁵. 9 peptides showed moderate to strong competitive binding (53-97.4% inhibition of the indicator EBV peptide) to DQ6 (FIG. 2A). These findings were generally consistent with in silico peptide binding predictions using the NetMHCIIpan3.2 software²⁶ (FIGS. 8A-8C). The 9 peptides span five regions (i-v) of prepro-HCRT. A strong binder (>75% competition), HCRT₁₋₁₅, was within the signal peptide region (i) and contained a 9-aa core, LPSTKVSWA (SEQ ID NO:31), previously shown to bind DQ6 by X-ray structure²⁷. Two overlapping peptides span the C-terminus of the signal peptide and N-terminus of the secreted HCRT1 neurotransmitter. This region (ii) contained 3 possible registers as predicted by NetMHCIIpan3.2 using nonamers (FIG. 8D). Using 15-mers, the algorithm only predicted 2 strong cores (FIG. 8C), and our empirical binding data further argued that the 9-aa core SSGAAAQPL (SEQ ID NO:32) present in the strong binder, HCRT₂₅₋₃₉, is the dominant register (FIG. 2A). Prepro-HCRT is processed intracellularly to two neurotransmitters, HCRT1 and HCRT2 known to interact with the HCRT receptors, HCRTR1 and HCRTR2, with different affinities²⁸. Interestingly, 5 of the DQ6-binding peptides were from highly homologous C-terminal region (iii) and region (iv) of the two processed neurotransmitters. Each region contains a NetMHCIIpan-predicted register (NHAAGILTL (SEQ ID NO:33) and NHAAGILTM (SEQ ID NO:34)), also implicated by empirical binding data (FIG. 2A). The ninth peptide was a weak binder (<75% competition) located at the prepro-HCRT C-terminal region (v), which is removed during processing to generate functional HCRT2. No strong binding register was predicted for this peptide (FIGS. 8C, 8D).

We further investigated core registers of the strong-binding peptides using X-ray crystallography. The structure of DQ6-HCRT₅₆₋₆₉ bound to DQ6 (Table 2) confirmed the predicted 9-aa core (NHAAGILTL (SEQ ID NO:33) as the correct binding register (FIG. 2B). Comparing the DQ6-HCRT₅₆₋₆₉ structure with the previously determined DQ6-HCRT₁₋₁₃ structure²⁷, we observed several conformational changes in α-helices of the DQ6α/β dimer (FIGS. 2C and 8E). However, the conformation of most of the DQ6 framework that would face a TCR was unchanged. We built on the structure of DQ6-HCRT₅₆₋₆₉ to model HCRT₂₅₋₃₇ and HCRT₈₇₋₁₀₀ binding to DQ6 (FIGS. 2D, 2E), and to infer candidate TCR-facing residues. Unlike the predicted TCR contact position P5K in HCRT₁₋₁₃, neither P5G in HCRT₅₆₋₆₉ nor P5G in HCRT₈₇₋₁₀₀ provided a side chain that could contribute to engagement with TCR, suggesting that TCR recognition of these complexes relied on P2/P3, or P8. HCRT₂₅₋₃₇ contained a DQ6-binding core with relatively short side chains at all TCR-facing residues: P2S, P3G, P5A, and P8P. Together, these findings predicted that DQ6-restricted TCRs might bind to multiple HCRT epitopes.

Various DQ6-HCRT_{peptide} Tetramers Stain CD4⁺ T Cells

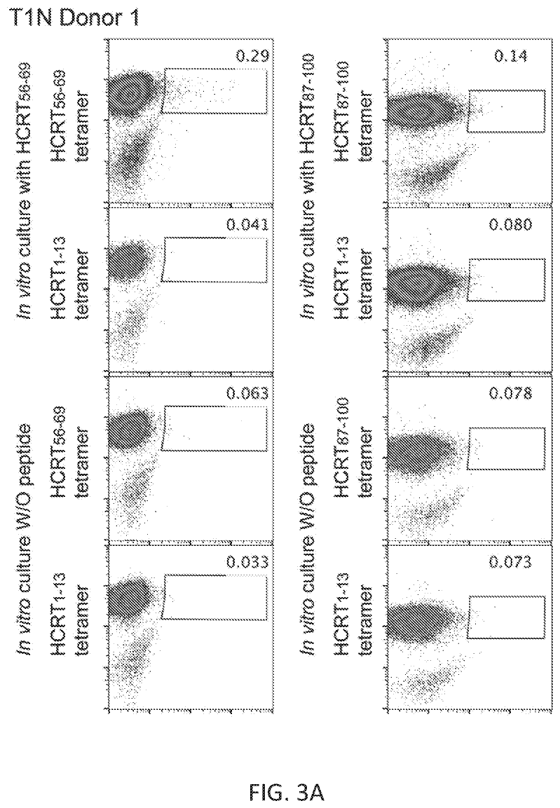

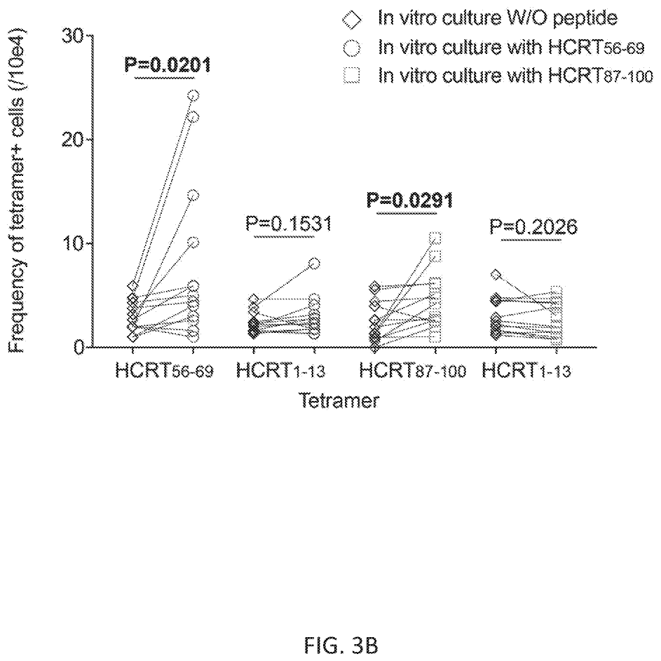

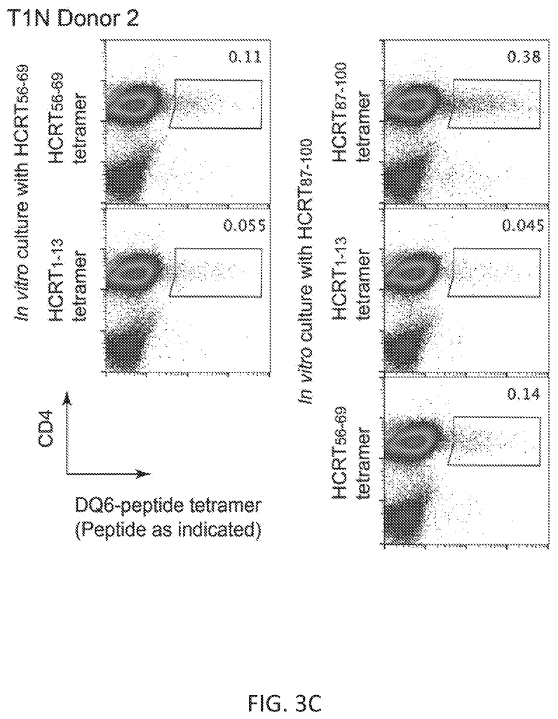

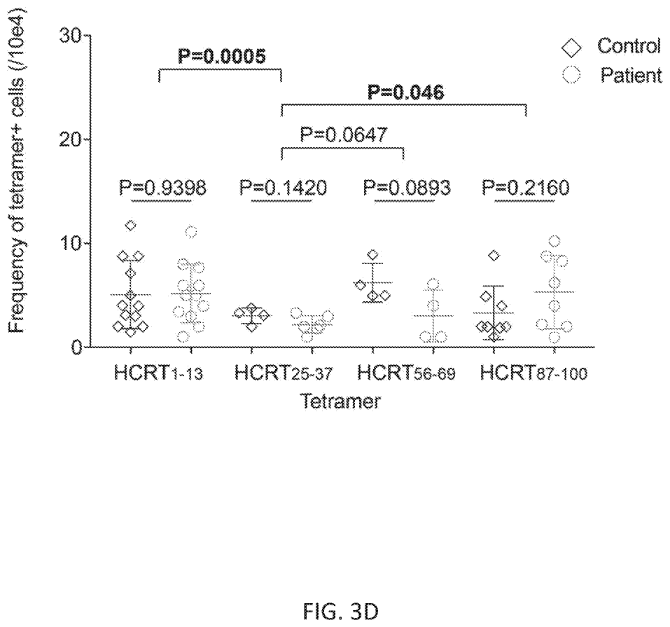

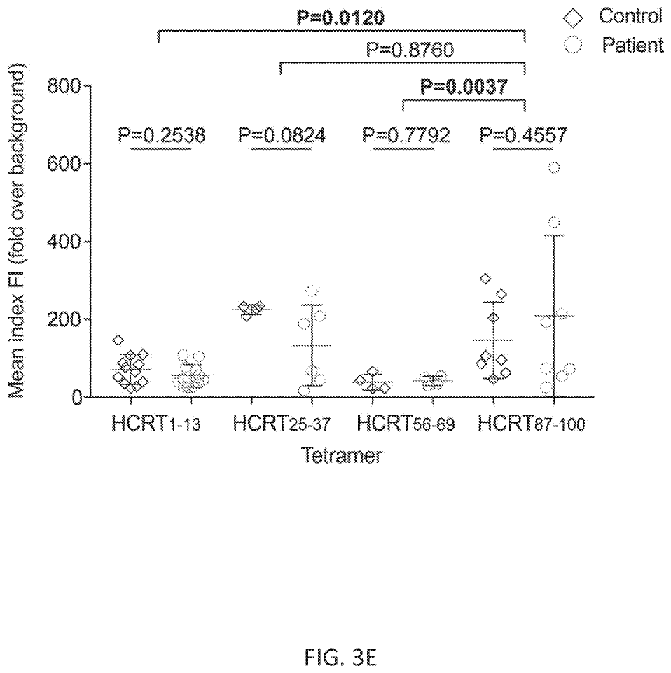

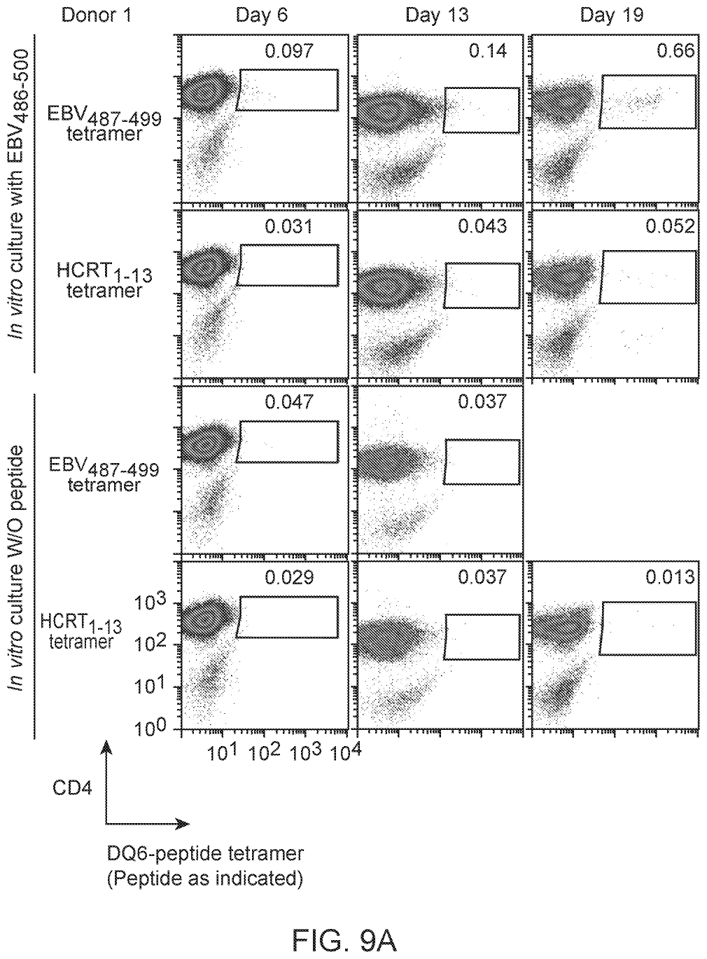

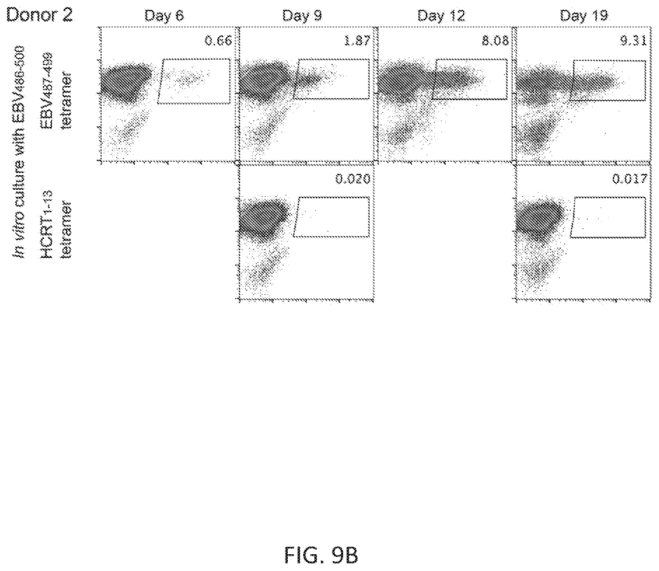

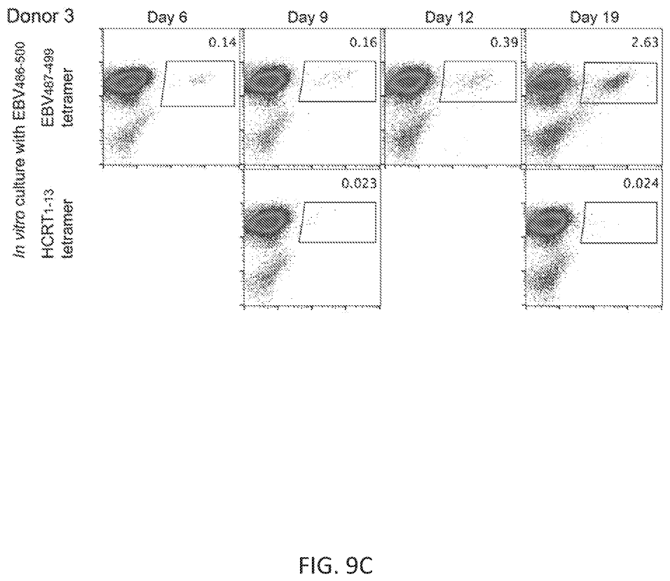

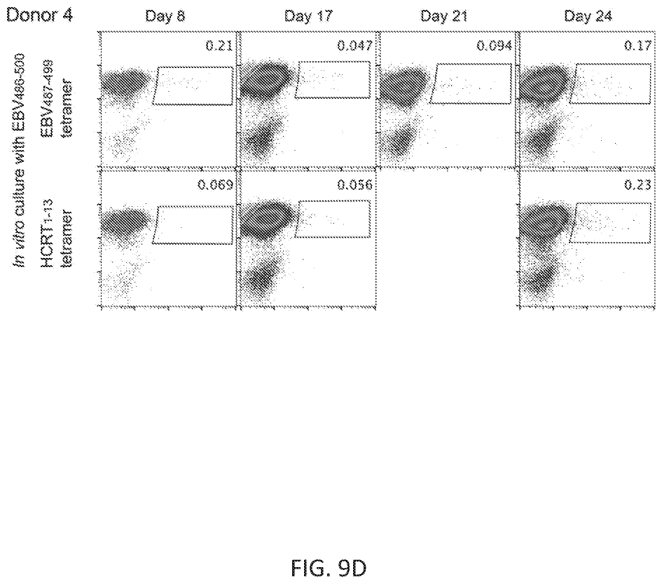

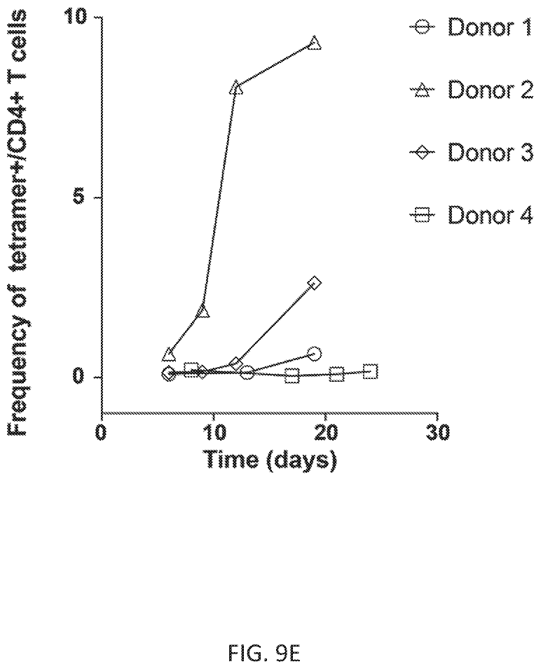

We next constructed four DQ6 tetramers using peptides with strong binding cores and tested their ability to stain CD4⁺ T cells isolated from peripheral blood mononuclear cells (PBMCs) of T1N donors. Tetramer-staining of T cells harboring HCRT-binding capability only rarely showed a discrete positive population (FIG. 3) like that seen with cells recognizing pathogen-derived epitopes such as EBV₄₈₆₋₅₀₀ (FIG. 9). The observed tetramer+/CD4⁺ T cell frequency of 0.039±0.0029% was consistent with a low frequency of circulating class II tetramer-positive cells, as previously described^{17,29-31}. Like DQ6-EBV₄₈₆₋₅₀₀ tetramer⁺ cells, which could be enriched in vitro (FIG. 9), frequencies of DQ6-HCRT₅₆₋₆₉ or DQ6-HCRT₈₇₋₁₀₀ tetramer⁺ cells from T1N donors were significantly increased after in vitro stimulation with the corresponding peptides (FIG. 3A, B), confirming the existence of circulating HCRT tetramer⁺ cells and the feasibility of using DQ6-HCRT_{peptide} tetramers to isolate potential autoreactive cells for further analysis. Cells enriched in vitro showed specificity for the peptide stimulator, as staining with HCRT₁₋₁₃ tetramer did not appreciably increase. In addition, however, we detected in vitro enrichment for DQ6-HCRT₅₆₋₆₉ tetramer⁺ cells in HCRT₈₇₋₁₀₀-stimulated cells from some T1N donors (FIG. 3C). This not only reflected the high homology between HCRT₅₈₋₆₆ and HCRT₈₉₋₉₇ registers, but also confirmed the structural prediction that some DQ6-restricted TCRs bind multiple epitopes from HCRT that result in signaling and cell expansion (FIG. 2E).

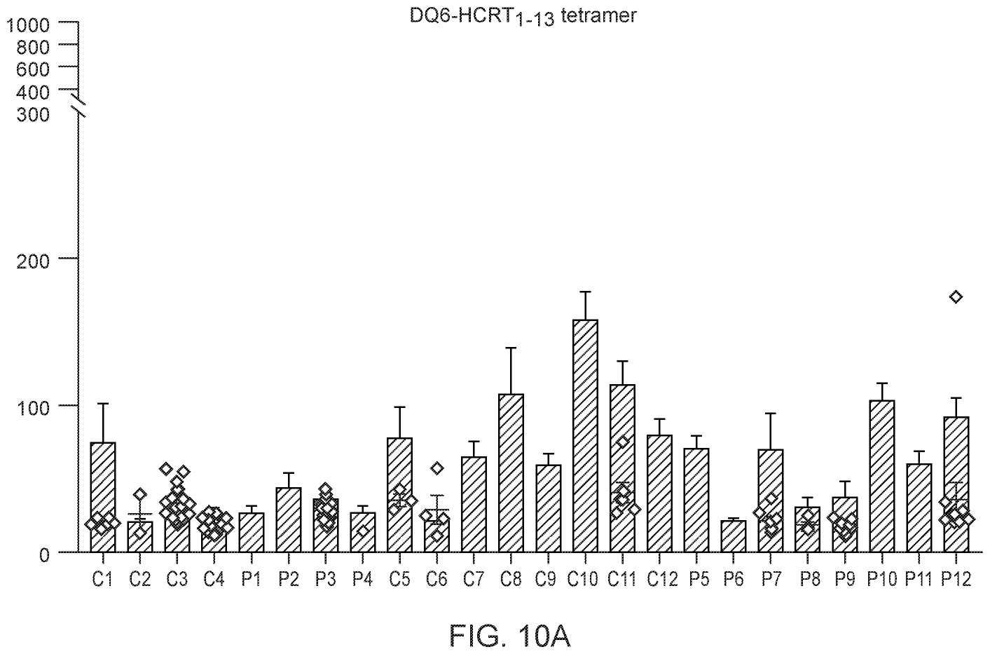

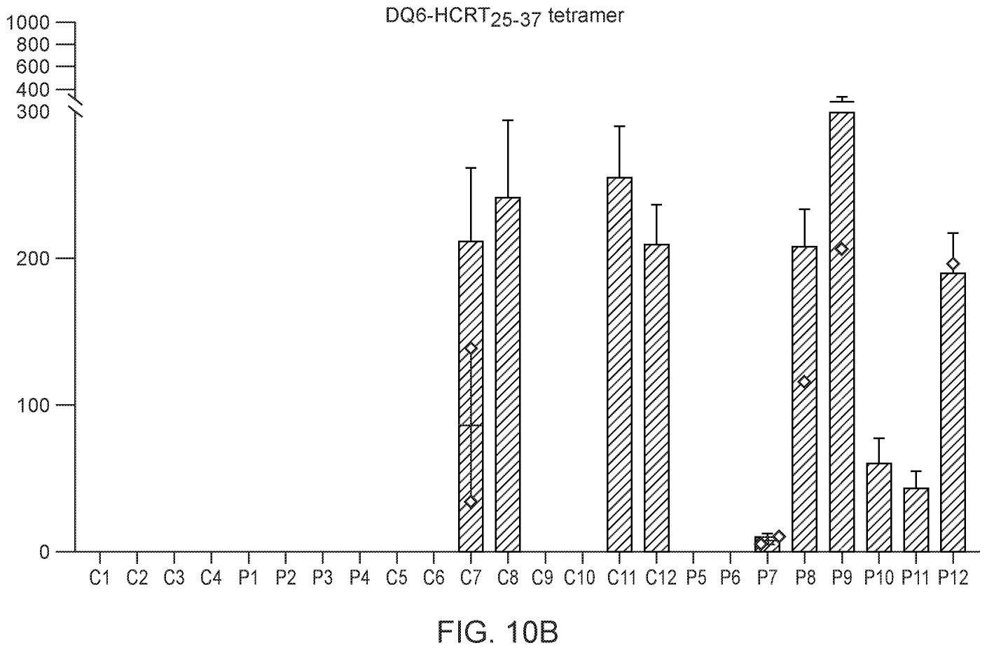

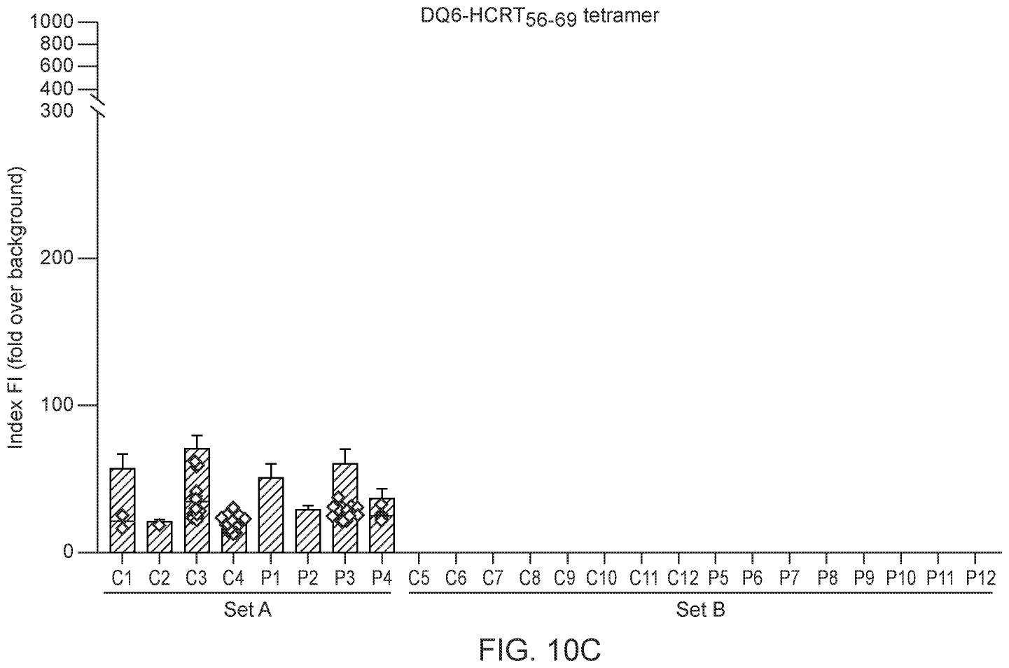

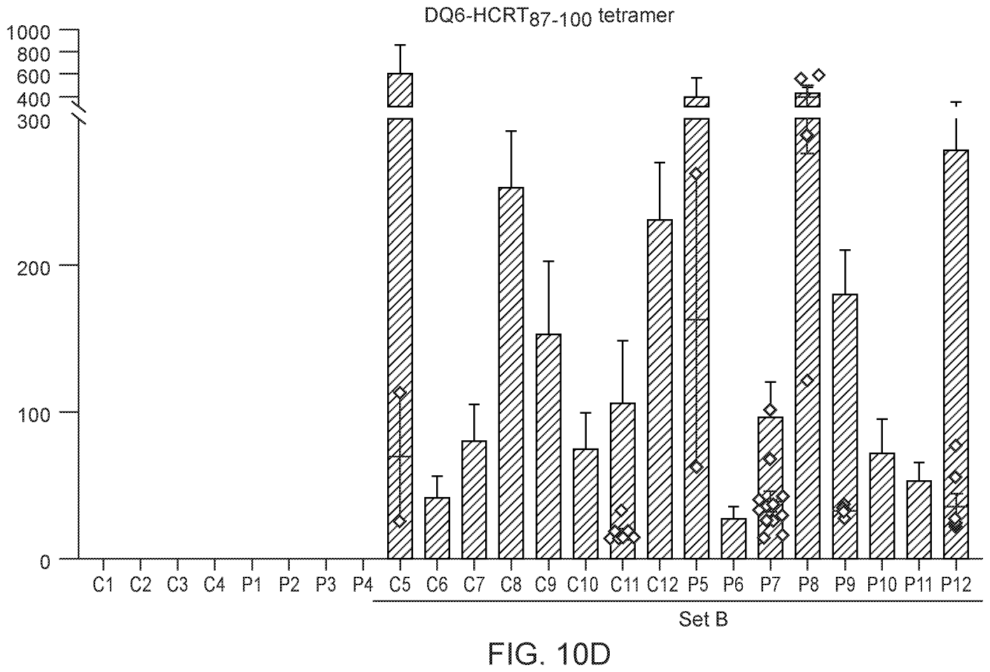

To get an unbiased report on in vivo clonal expansion of circulating DQ6-HCRT_{peptide} tetramer⁺ cells and to preserve their phenotypes, we sorted unmanipulated ex vivo DQ6-HCRT_{peptide} tetramer⁺/CD4⁺ T cells from T1N and control DQ6⁺ donors and examined physiologic antigen-driven expansion by single-cell sequence analysis. Prior to sequence analysis, we performed an overall comparison of the different DQ6-HCRT_{peptide} tetramer⁺/CD4⁺ cells from DQ6⁺ patients and controls. Notably, the DQ6-HCRT₂₅₋₃₇ tetramer yielded significantly fewer tetramer⁺ cells when compared with the other 3 DQ6-HCRT_{peptide} tetramers (FIG. 3D). As single cell index sorting (iFACS)³² records fluorescence intensity (FI) parameters of individual cells (see methods), we used mean index Fl of tetramer binding signal to rank tetramer⁺ cells. We found that DQ6-HCRT₈₇₋₁₀₀ tetramer⁺ cells showed the largest range of signal intensity among cells from different donors (especially T1N patients), and the average binding signal was significantly higher than DQ6-HCRT₁₋₁₃ or DQ6-HCRT₅₈-s₉ tetramer⁺ cells (FIGS. 3E and 10), raising the possibility of escape from tolerance mechanisms of diverse affinity clonotypes specific for the epitope in HCRT₈₇₋₁₀₀ among DQ6+ donors, especially T1N patients.

### Potential T1N-Association of the Epitope in HCRT₈₇₋₁₀₀

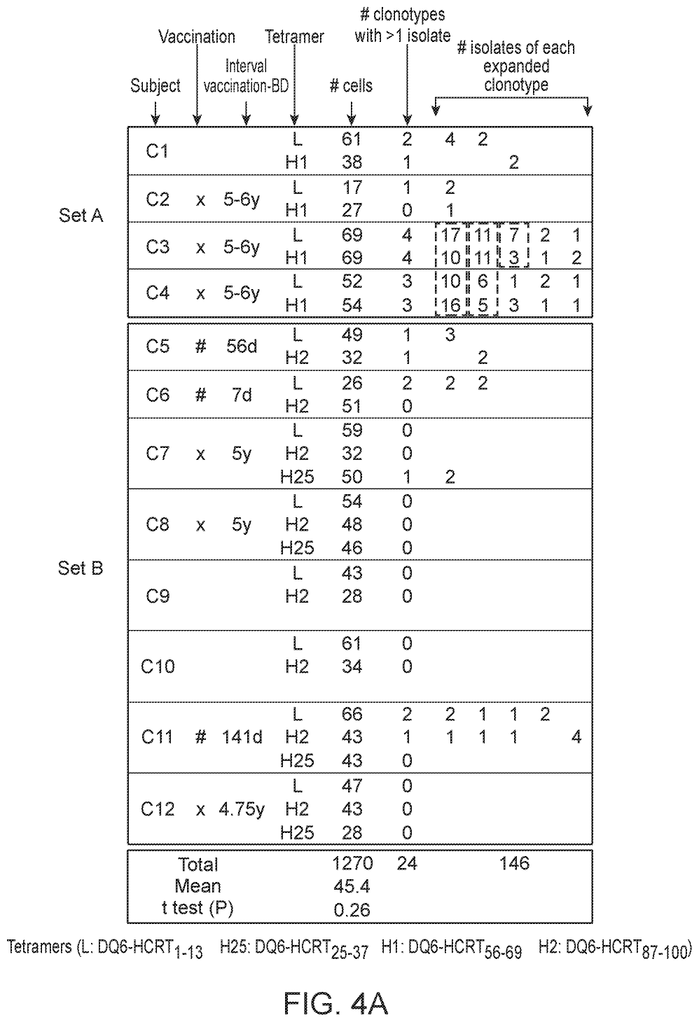

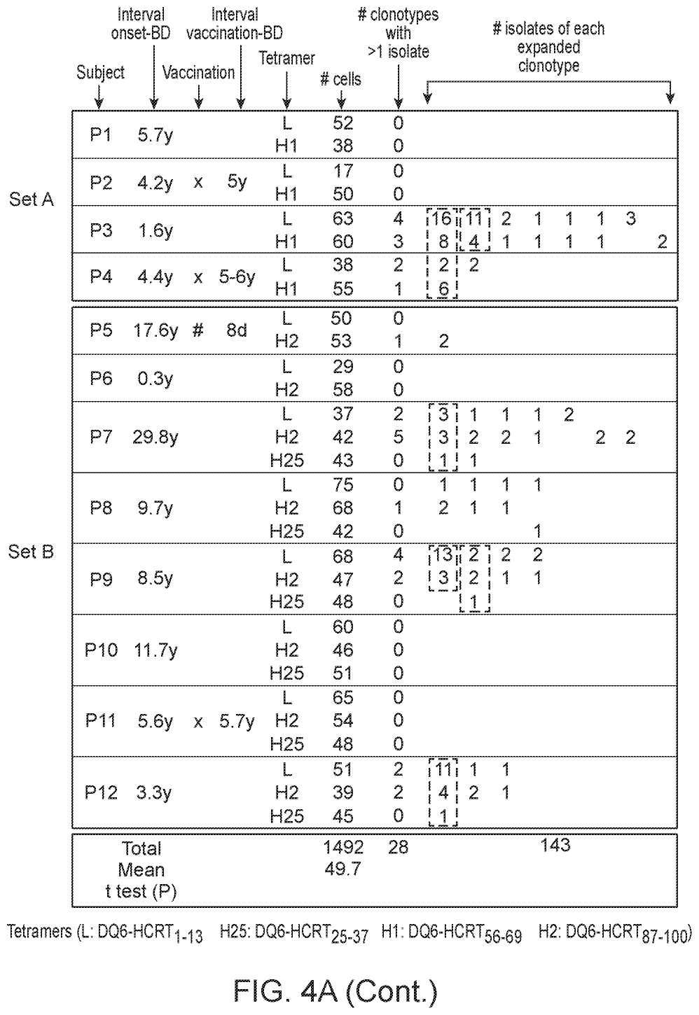

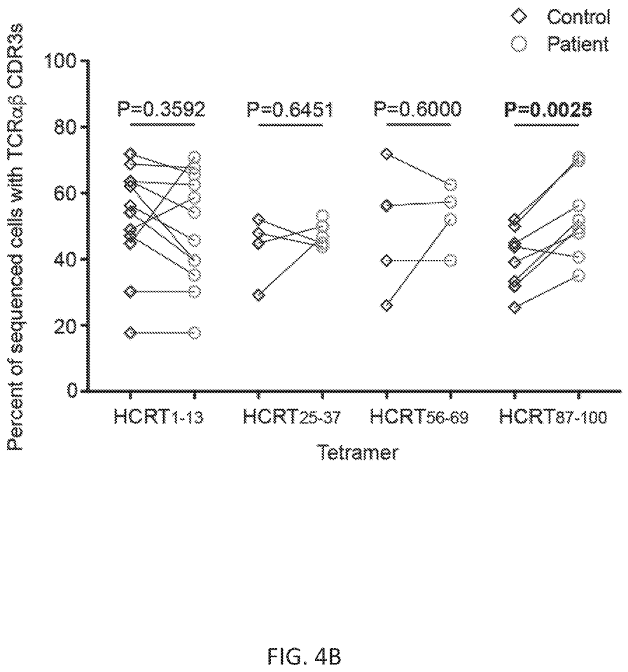

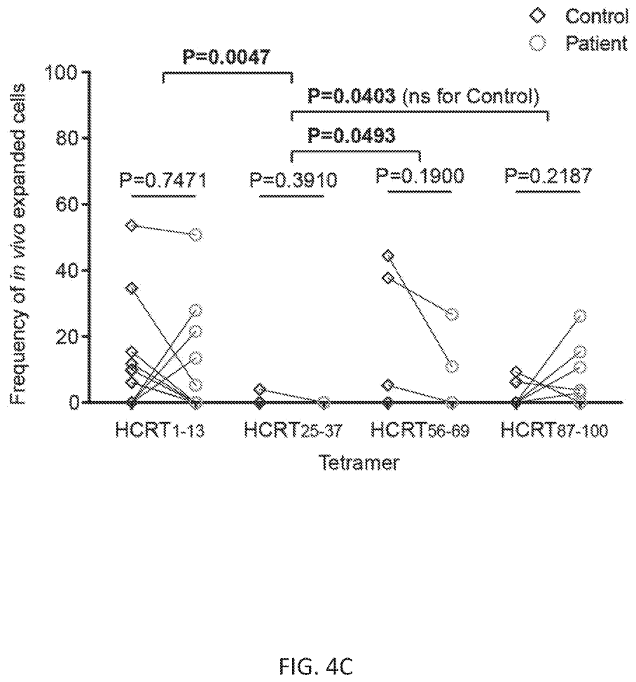

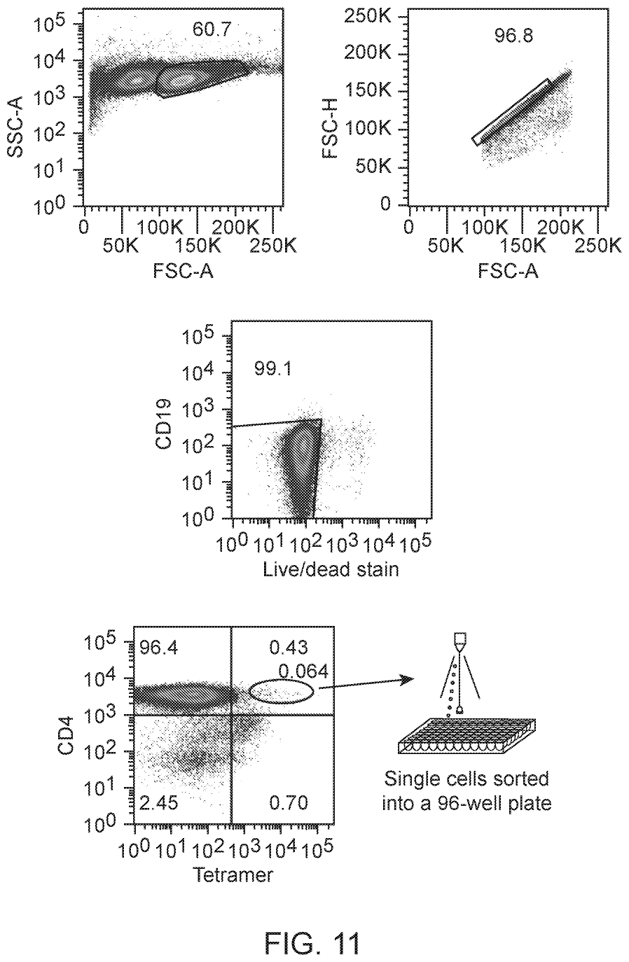

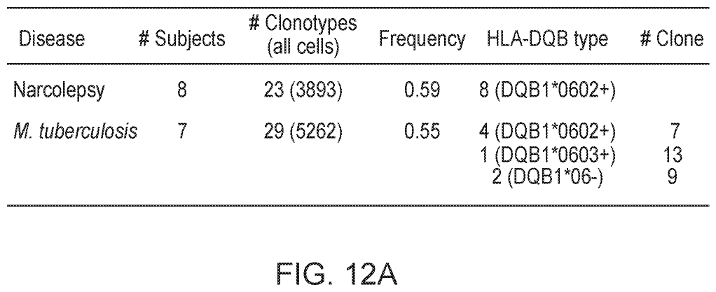

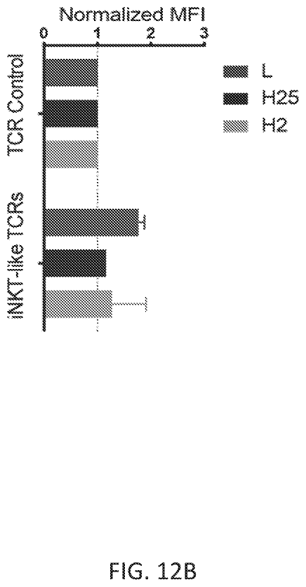

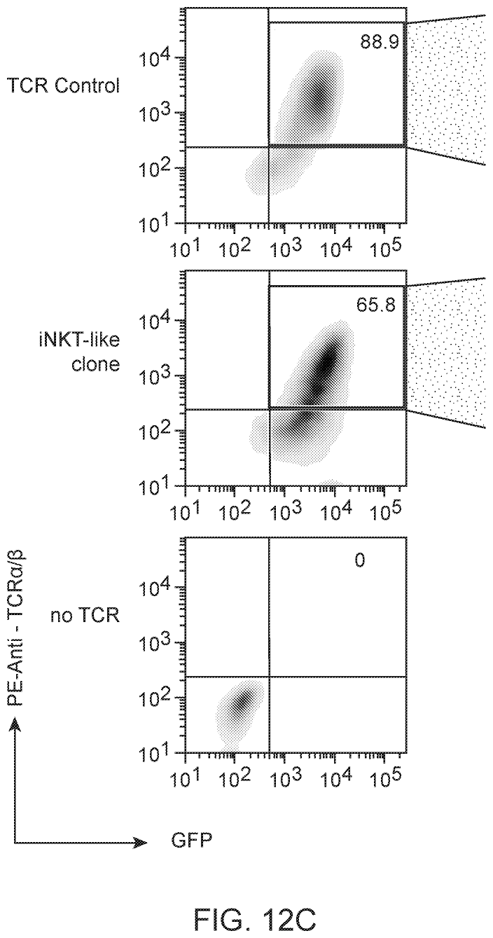

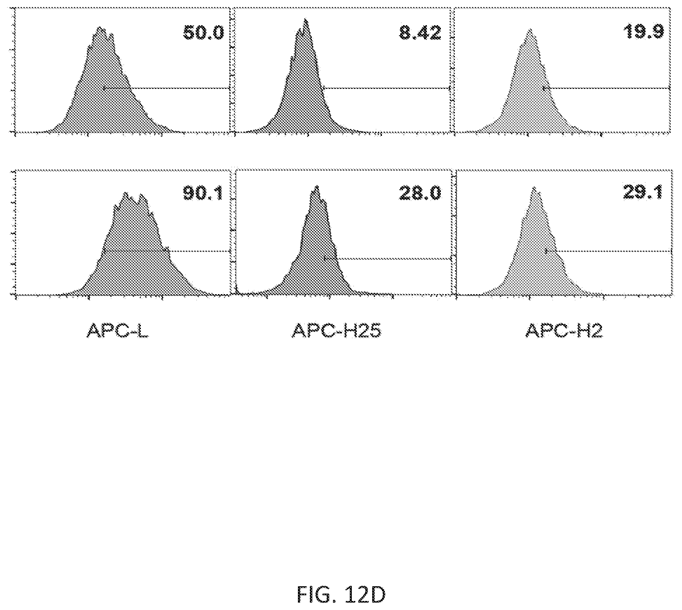

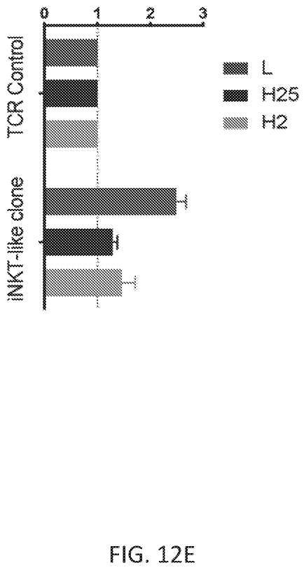

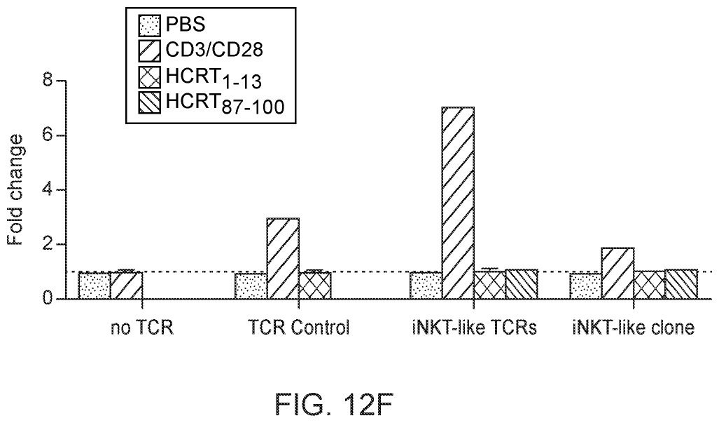

We observed DQ6-HCRT_{peptide} tetramer⁺/CD4⁺ cells in case and control cohorts of DQ6⁺ individuals, which is not surprising considering the breadth of TCR binding capacity captured by tetramers (FIGS. 3D, 3E). Taking advantage of this, we successfully deep sequenced comparable numbers of tetramer⁺/CD4⁺ single cells from both cohorts. iFACS screening of 3-5 million CD4⁺ T cells from each donor was necessary for the isolation of one 96-well plate of single cells (FIG. 11). We used blindly paired case/control PBMC samples for each experiment (see FIG. 4A) to reduce effects of technical variations on downstream analyses of case/control differences. In total, 5503 wells of sorted individual cells were analyzed using the established algorithm¹⁹. TCR transcripts were detected in 4605 wells (83.7% well coverage); as commonly observed with this approach^{19,20,29}, not every well yields called TCRα/β from raw sequence reads. Out of 2762 sequenced cells that had paired TCRα/β and productive α/β CDR3 sequences, 1492 cells were from 30 case plates and 1270 cells were from 28 control plates (FIG. 4A), indicating the absence of technical bias towards either cohort. However, unique to DQ6-HCRT₈₇₋₁₀₀ tetramer⁺/CD4⁺ cells, 7 out of 8 cases yielded significantly (**P=0.0025) more called TCRα/β among sorted cells compared to their paired controls (FIG. 4B). This systematic increase indicates a biological signal that survives technical variations inherent in independent sort/sequence experiments of case/control pairs and implies a significant change in the repertoire of circulating T cells in T1N patients, in favor of available DQ6-HCRT₈₇₋₁₀₀ tetramer⁺/CD4⁺ cells for detection.

As in vivo antigen-driven clonal expansion changes the circulating T cell repertoire and is a feature of disease-related clones, we next identified and analyzed expanded clonotypes among the single cell sequences. Overall, 52 TCRα/β clonotypes (˜1% of all sorted cells) had more than one isolate. 5 additional clonotypes were identified in two different subjects, but only once each (i.e., unexpanded public clonotypes). Similar to the overall low frequency of DQ6-HCRT₂₅₋₃₇ tetramer⁺ cells (FIG. 3D), DQ6-HCRT₂₅₋₃₇ tetramers detected significantly fewer expanded clonotypes (1 from a control C7 out of 443 total) compared to the other tetramers (FIG. 4C). These results are consistent with HCRT₂₅₋₃₇ peptide rarely being presented in vivo, likely due to it spanning a border between the signal peptide and HCRT1 and harboring a site for proteolytic cleavage. In contrast, DQ6-HCRT₈₇₋₁₀₀ tetramers identified expanded clonotypes in 5/8 cases (highly expanded clonotypes with >5 isolates seen in 3 cases: P7, P9, and P12) vs 2/8 controls (no highly expanded clonotypes). Similar case/control differences were not observed in the expanded clonotypes isolated by other tetramers (FIG. 4A). Particularly in T1N patients P7-12, there were significantly more expanded clonotypes identified by DQ6-HCRT₈₇₋₁₀₀ tetramer than by DQ6-HCRT₂₅₋₃₇ tetramers (8% vs 0%, FIG. 4C). Collectively, the above findings argued that the recognition of epitope in HCRT₈₇₋₁₀₀ by CD4⁺ T cells may be T1N-associated.

Interestingly, 34 out of the 52 expanded TCR clonotypes were identified by more than one DQ6-HCRT_{peptide} tetramer (FIG. 4A). This high frequency was reminiscent of the implication of possible cross-reactive binding from our structural analysis. Although cross-reactive binding may not trigger TCR signaling, our observation of many clonotypes with cross-reactive potential raised the possibility that some in vivo clonal expansion was triggered by unknown self/foreign epitopes and perhaps related to T1N risk, but not disease. Indeed, although in vivo expansion of cross-reactive clonotypes was observed in similar numbers of controls (4/12: C2-4 and C11) and cases (6/12: P3, P4, P7-9, P12), 4/4 controls, but only 1/6 cases (P4), were subjects who had received influenza vaccination (FIG. 4A). This finding did not conflict with the reports that H1N1 infection or vaccination promoted T1N during the 2009 flu pandemic^{33,34}, as many cases studied here did not develop in that time period. Instead, this finding strengthened our argument that the epitope in HCRT₈₇₋₁₀₀ may be T1N-associated, as none of the 4/8 cases (P7-9 and P12) harboring DQ6-HCRT₈₇₋₁₀₀ tetramer⁺ cross-reactive clonotypes was previously H1N1 influenza-vaccinated, whereas controls harboring expanded and/or cross-reactive clonotypes identified by DQ6-HCRT₈₇₋₁₀₀ tetramers were all H1N1-vaccinated (FIG. 4A).

TCRs with a T1N Signature Respond to the Epitope in HCRT₈₇₋₁₀₀

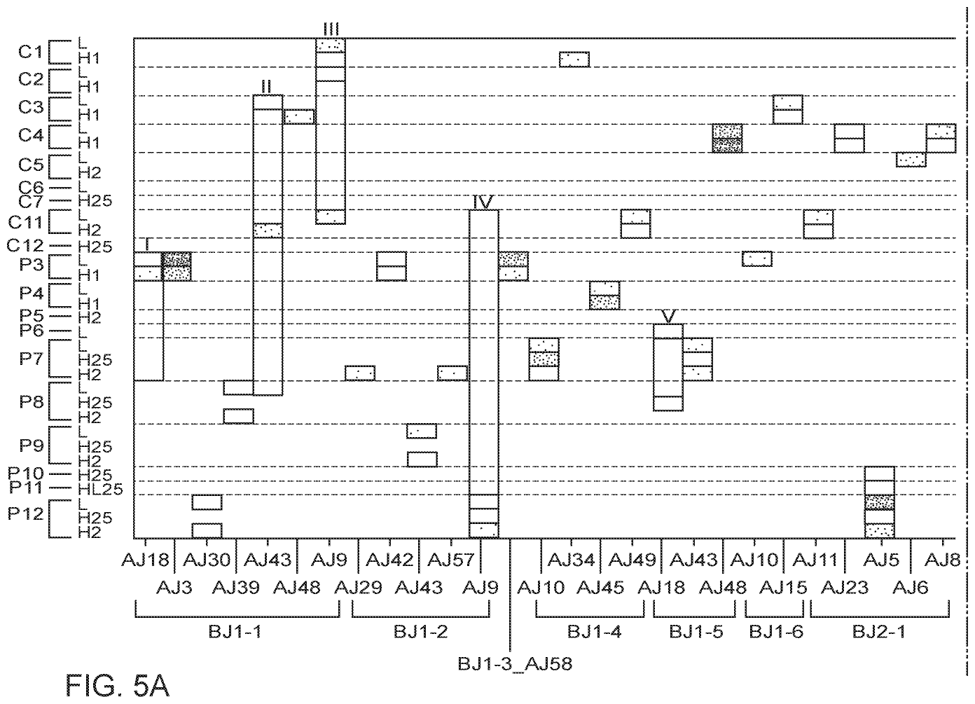

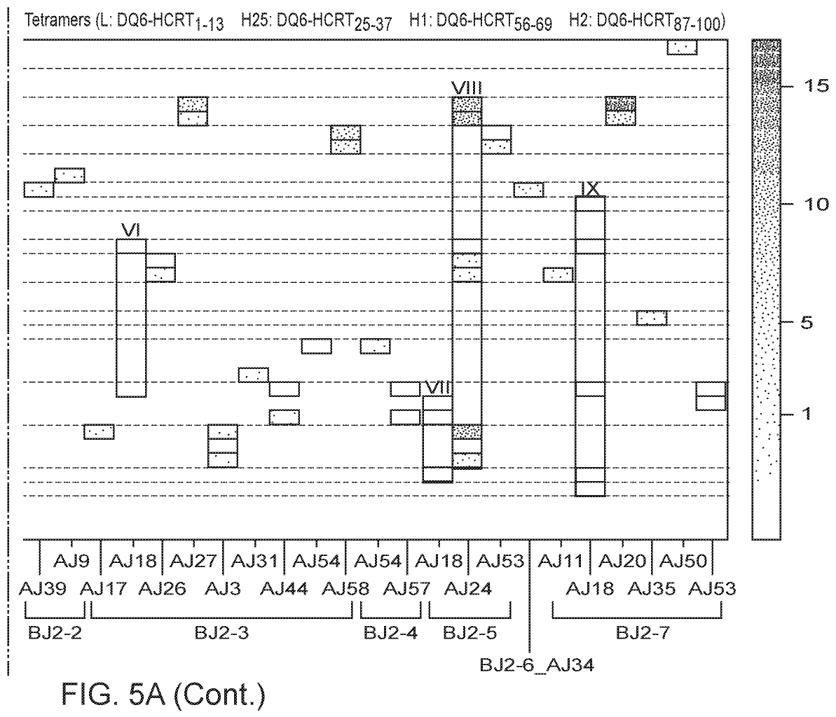

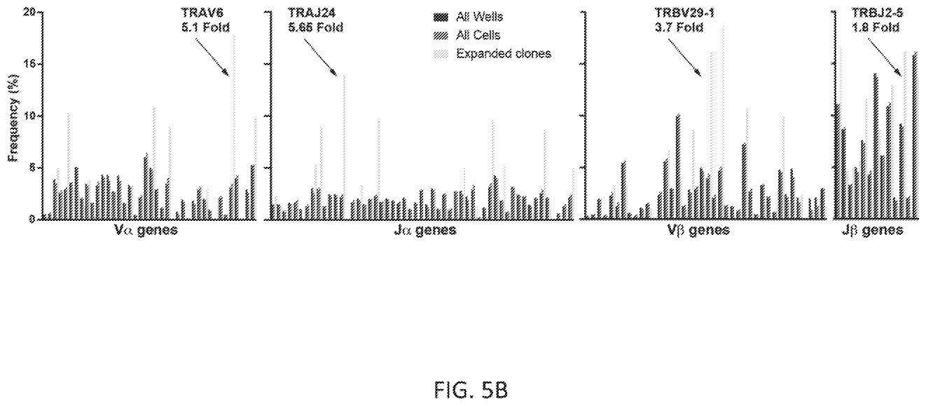

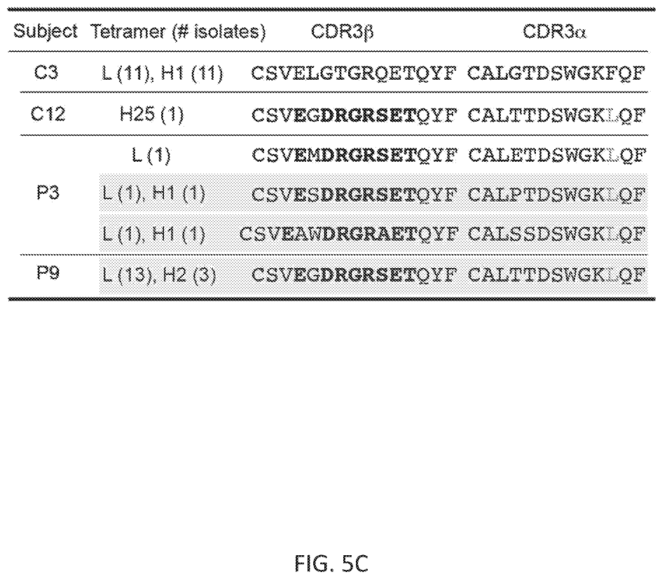

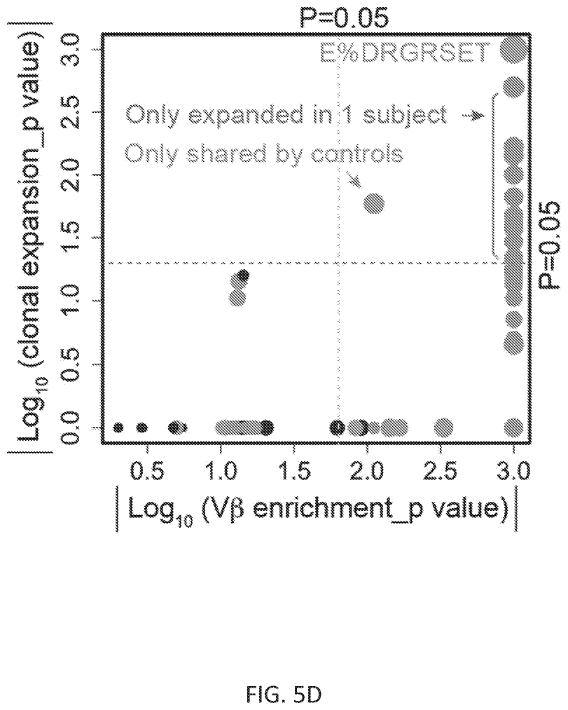

To search for a T1N signature in cells stained by DQ6-HCRT_{peptide} tetramers, we first compared Jα/Jβ gene usage in 57 TCRs clonotypes observed more than once in our dataset (302 cells covering all expanded and unexpanded public clones). We found 9 (1-IX) groups of cells isolated from different subjects that express identical TRBJ/TRAJ genes (FIG. 5A). These 9 groups covered 3 major types of TCRs: Type A contained non-public CDR3s using varied V genes, although J genes are the same; Type B used public CDR3a encoded by TRAV10_J18 but mostly different CDR3P encoded by TRBV25-1 in combination with varied JP genes, a sequence signature reminiscent of the one observed in semi-invariant natural killer T (iNKT) cells³⁵; Type C used conserved/semi-public α/β CDR3s encoded by identical V-J genes. The single-cell sequencing technique is unable to estimate an overall frequency of α/β paired CDR3s given the limitation of 50-150 testable tetramer⁺/CD4⁺ single cells per subject. However, within the limited number of sequences from 302 cells, 43 of 79 total type C cells expressed highly conserved clonotypes using paired TRBV29-1_J2-5/TRAV6_J24 genes (42 expanded from C3, P3, and P9; 1 unexpanded public from C12, all in group VIII). The gene frequencies in these 302 cells were significantly different from those in 2762 cells with paired α/β CDR3s or in 4605 wells that returned any TCR sequence (FIG. 5B). Therefore, the skewing of gene usage in these 302 cells, for example, a 5.65-fold increase of TRAJ24, was not related to sequencing bias, but rather a reflection of in vivo expansion and/or some public features of certain clonotypes.

We then focused on CDR3s in all clonotypes from our dataset using TRBV29-1_J2-5/TRAV6_J24 genes, a total of 44 cells including another unexpanded clonotype from P3. All a chains used conserved CDR3 sequences, CALxTDSWGKF(L)QF (FIG. 5C). For all TRAJ24-expressing TCRα CDR3 sequences from our dataset, we determined the SNP allele, either TTC or TTG, encoding F/L. The striking finding was that in vivo expansion of clonotypes carrying the risk allele, TRAJ24L, was only observed in the two cases (P3 and P9). These expanded risk-bearing clonotypes (eTRAJ24L hereafter) shared very similar binding features, as these cells were isolated by binding to both DQ6-HCRT₁₋₁₃ and DQ6-HCRT₅₆₋₆₉/HCRT₈₇₋₁₀₀ (epitopes in HCRT₅₆₋₆₉ and HCRT₈₇₋₁₀₀ share high homology) tetramers. Also astonishing was that the β chains paired with the α chains in these 3 clonotypes also used very conserved CDR3 sequences, CSVExDRGRSETQYF (SEQ ID NO:36, FIG. 5C). A novel motif analysis tool, GLIPH²⁰ suggested that out of 181 CDR3P motifs found in our dataset, the global motif (E%DRGRSET, % allows varied residues) shared by many TCRs from cases and controls had an overall significance (final score) that was over two orders of magnitude higher than any other motif (FIG. 5D). In contrast, although expanded (either T1N-irrelevent or suppressive), the clonotype using TRBV29-1_J2-5/TRAV6_J24 genes in C3 was composed of less conserved CDR3P paired with the non-risk TRAJ24F-bearing CDR3a. Notably the highly expanded eTRAJ24L clonotype from patient 9 (TCR27 hereafter), identified by both DQ6-HCRT₁₋₁₃ and DQ6-HCRT₈₇₋₁₀₀ tetramers, was composed of a public CDR3a observed in P7, P9, P12 and C12; and a public CDR3P observed in P8, P9, P10, C11 and C12. The sharing of public features across multiple donors might have increased the chance for detection of TCR27 even when the cell expressing them was unexpanded. The DQ6-HCRT₂₅₋₃₇ tetramer isolated an unexpanded solo cell expressing identical α/β CDR3 nucleotide sequences as TCR27 from C12, who carries the risk SNP allele (FIG. 5C). Notably, index Fl analysis showed an intermediate tetramer binding rank for cells expressing eTRAJ24L clonotypes (FIG. 5E), as might be expected for an autoreactive clonotype, which survived thymic selection through low to moderate affinity³⁶ and expanded in vivo. The sequence analysis suggested that clonally expanded cells expressing particularly paired TCRα/β, harboring TRAJ24L, likely contribute to T1N development.

eTRAJ24L-Mediated Signaling Against the Epitope in HCRT₈₇₋₁₀₀

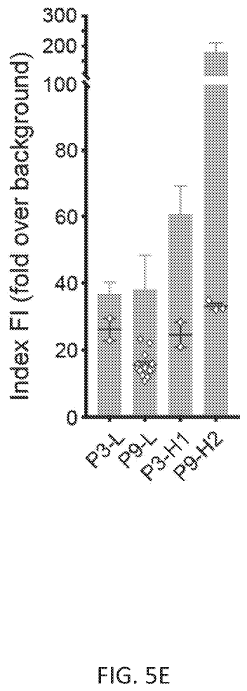

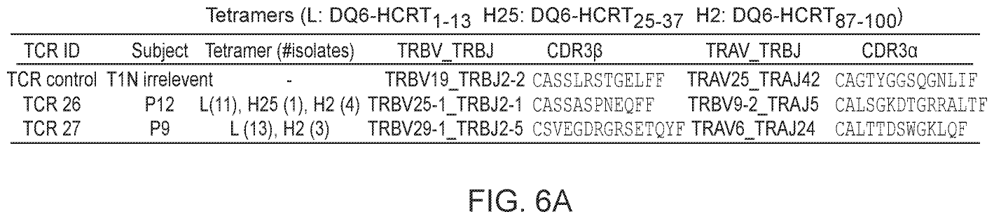

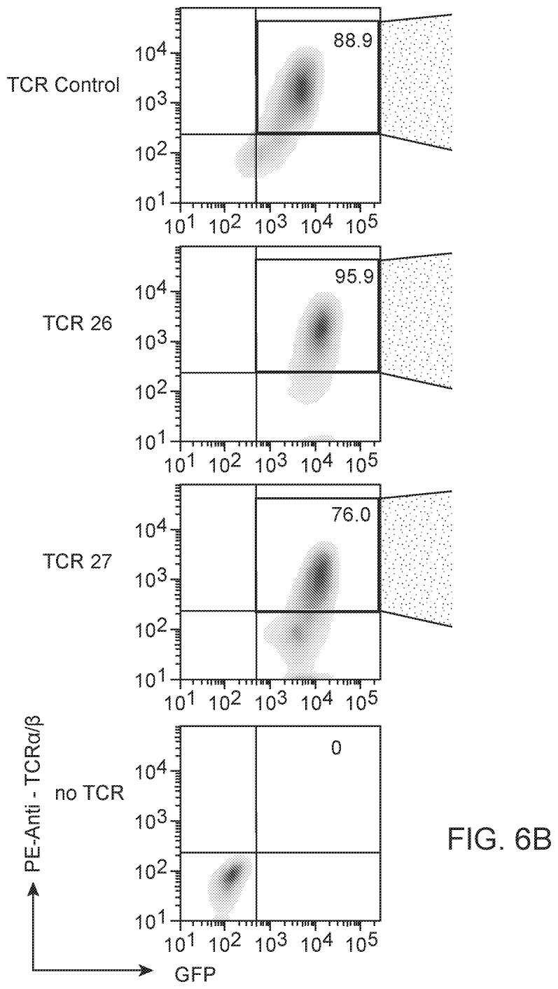

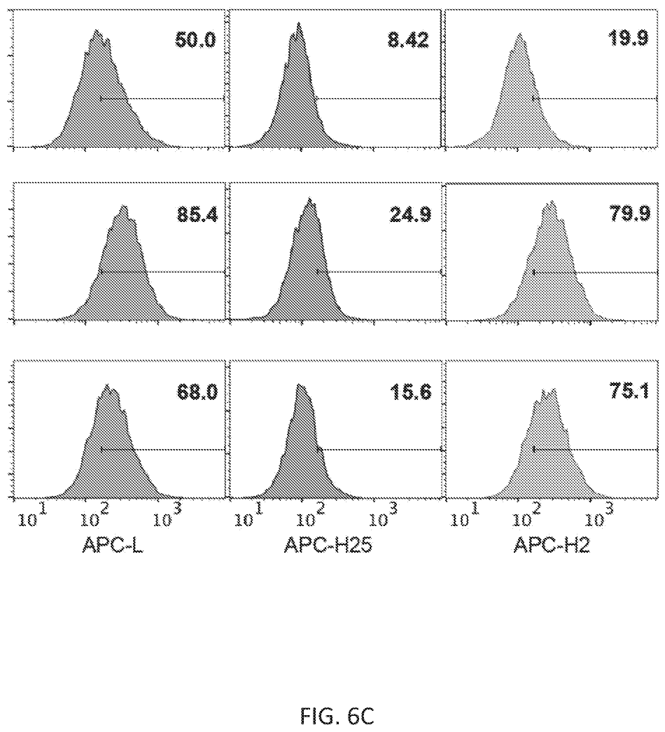

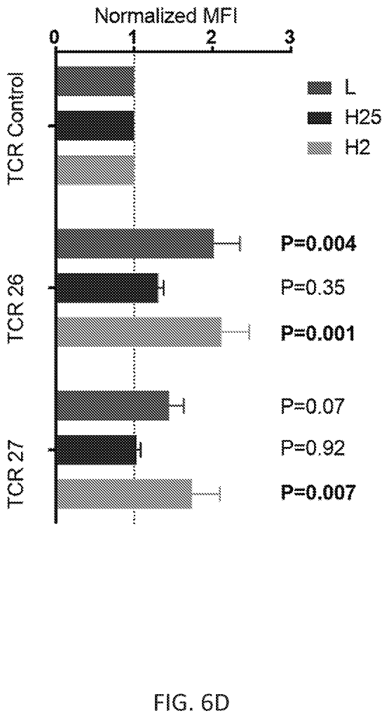

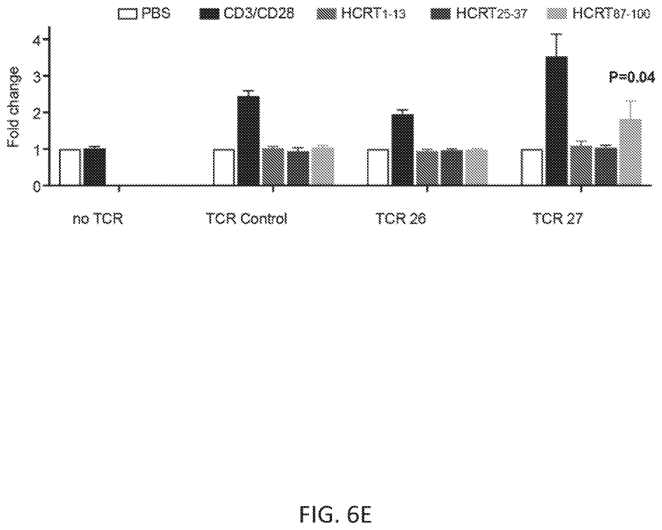

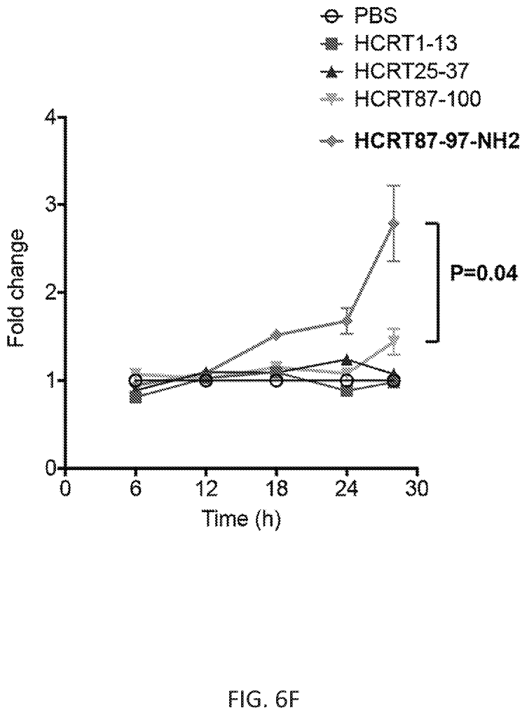

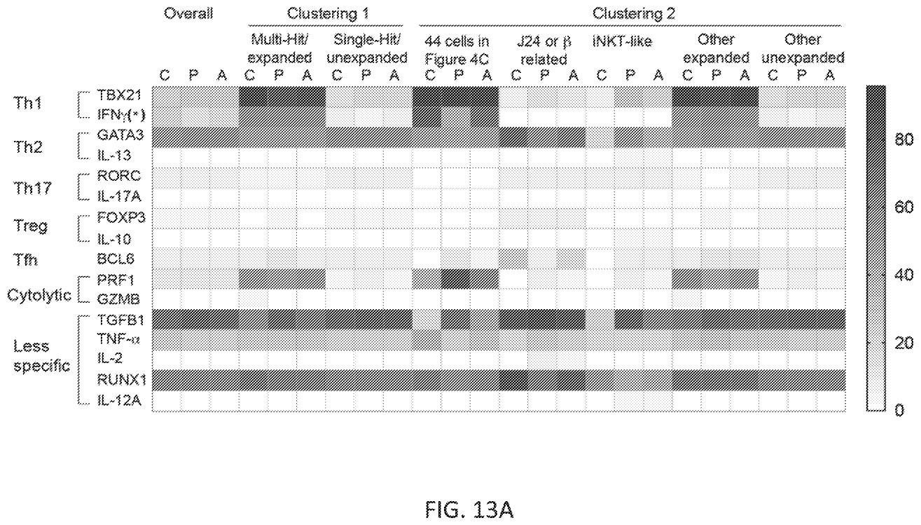

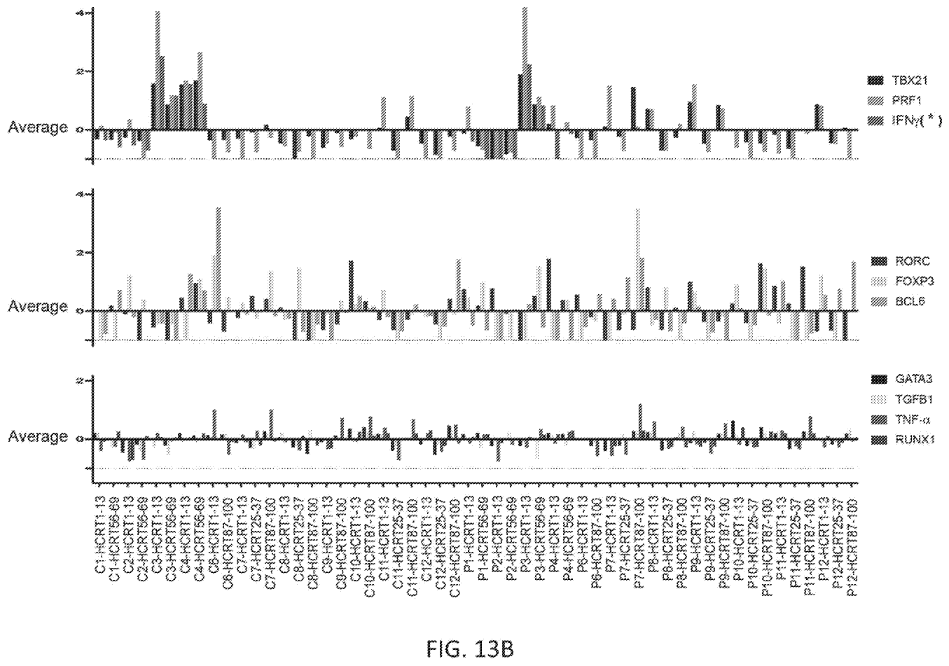

To further examine HCRT peptide-specific binding and signaling of eTRAJ24L clonotypes, we expressed TCR27 in a TCRα/β-deficient Jurkat cell line. We also generated transfectants for comparison: one with a patient-derived non-TRAJ24 expressing clonotype (TCR26), which contains a second frequently used CDR3P motif (FIG. 5D); one with a T1N-irrelevant TCR isolated from a CD8⁺ T cell; others with tetramer-identified TCRs bearing iNKT-like signatures. We reconstructed TCRs using sequence-determined α/β CDR3 nucleotides in frame with the germline sequences of the identified gene segments (IMGTN-QUEST³⁷) (FIGS. 6A and 13A). Flow cytometric analysis confirmed the surface expression of these TCRs in the Jurkat transfectants (FIGS. 6B and 13D). Compared to the irrelevant control, TCR27, TCR26 and iNKT-like TCRs showed appreciably more TCR⁺/tetramer⁺ cells and relatively higher staining signals of HCRT₁₋₁₃ or HCRT₈₇₋₁₀₀ tetramers (FIGS. 6C, 6D and 13C, 13E, 13F), consistent with single cells expressing these TCRs being isolated by these tetramers. The difference from control (˜1.5-2.5 fold) in MFI was not dramatic, consistent with the moderate tetramer binding rank of these TCRs, as determined by iFACS (FIGS. 5E and 10).

We next tested activation of these TCR transfectants in response to DQ6-restricted presentation of a HCRT epitope. TCR transfectants were incubated with control stimuli or HCRT peptides presented by an APC cell line that expresses DQ6 as the only HLA class II molecule (see methods). All transfected TCRs transmitted a CD3/CD28-mediated signal, indicating functional CD3 co-expression with TCR on the surface of transfectants. Very strikingly, only DQ6-restricted presentation of HCRT₈₇₋₁₀₀ peptide to TCR27 was able to trigger TCR-mediated activation signaling out of all HCRT peptide/TCR pairs tested using the validated Jurkat-reporter system²⁰ (FIGS. 6E and 13G). As the physiological amidation process converts the C-terminal Gly98 of HCRT2 neurotransmitter to a C-terminal amide (—NH2)²⁸, the likelihood of HCRT₈₇₋₉₇-NH2 being presented by DQ6 may be higher than HCRT₈₋₁₀₀ peptide. Consistent with this hypothesis, we found that the HCRT₈₇₋₉₇-NH2 triggered stronger signaling in TCR27 transfectants than did HCRT₈₇₋₁₀₀ peptide using the Jurkat-reporter system (FIG. 6F). These results argue that epitope in HCRT₈₇₋₉₇ contributes to the in vivo clonal expansion of TCR27⁺ cells in the T1N patient.

Phenotypic Features Linked with eTRAJ24L

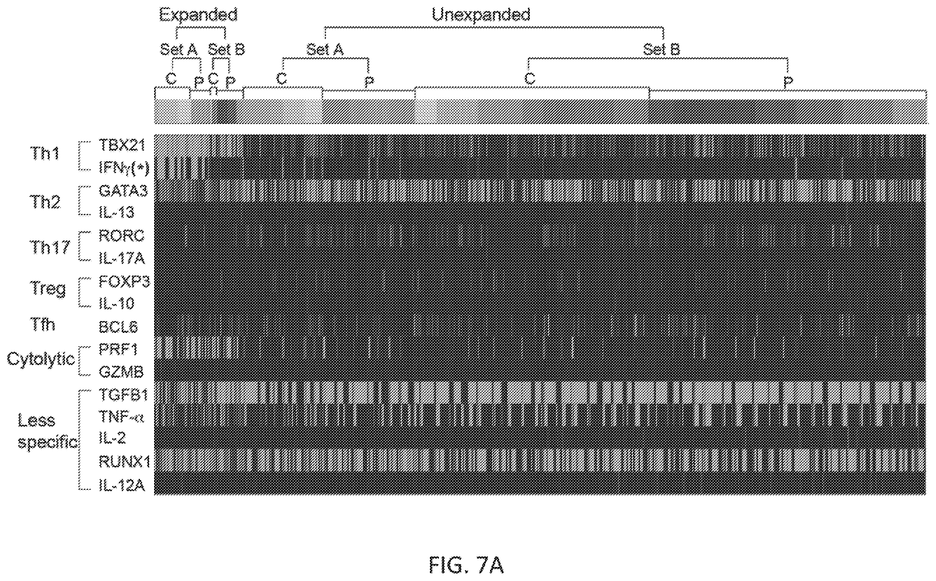

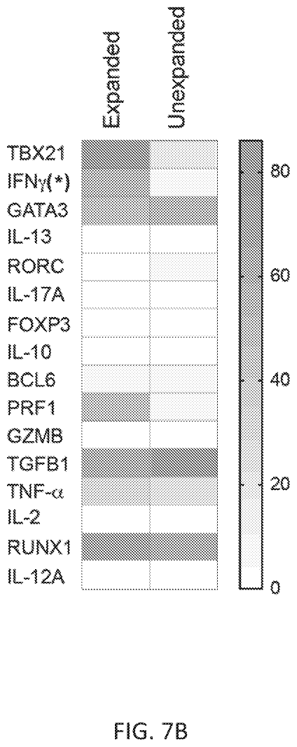

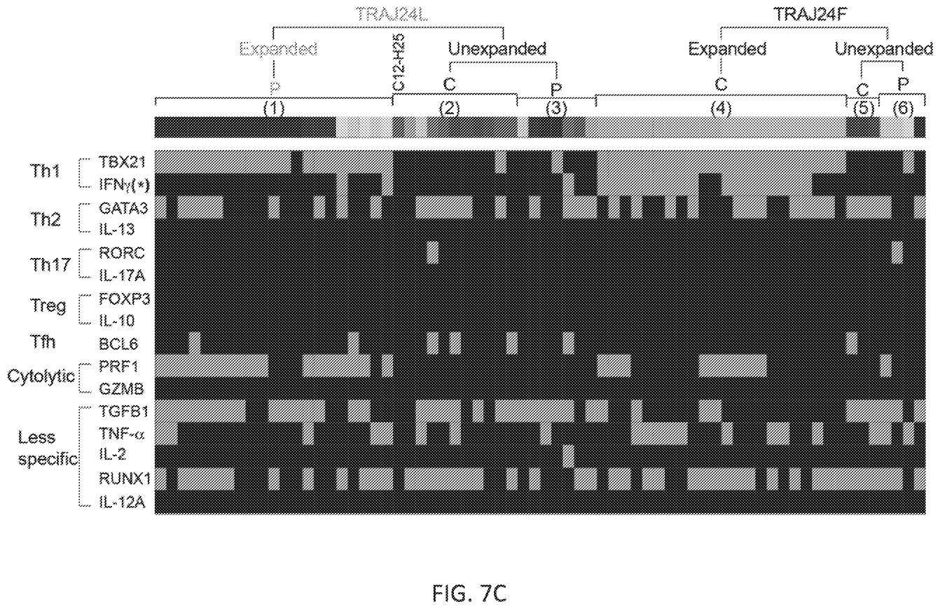

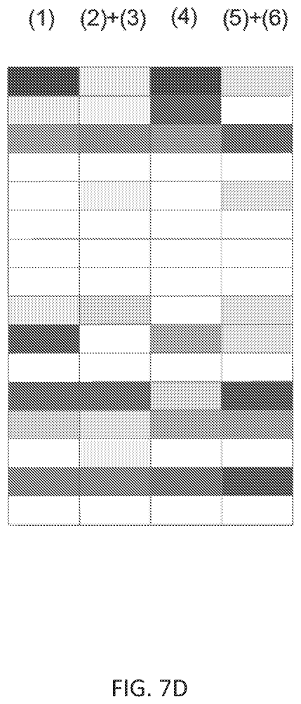

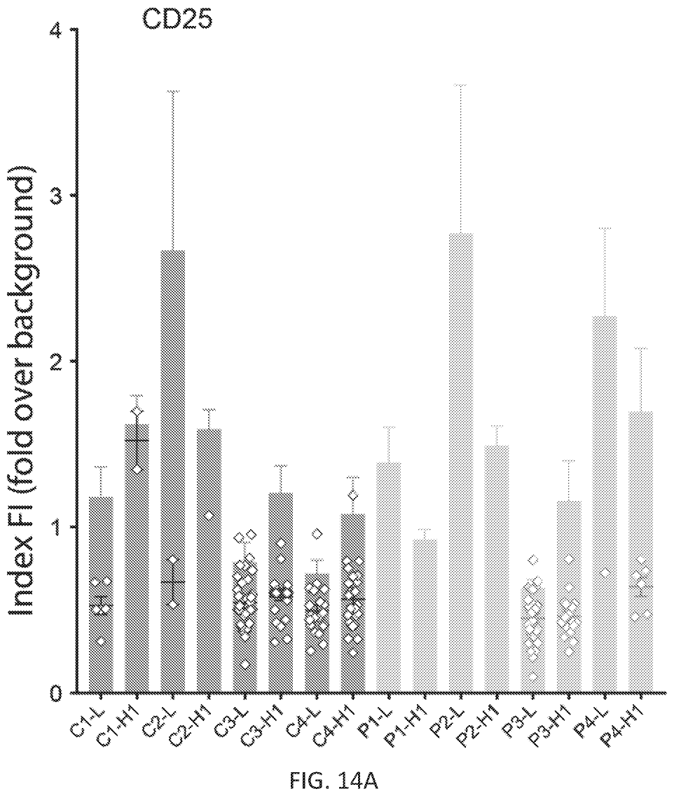

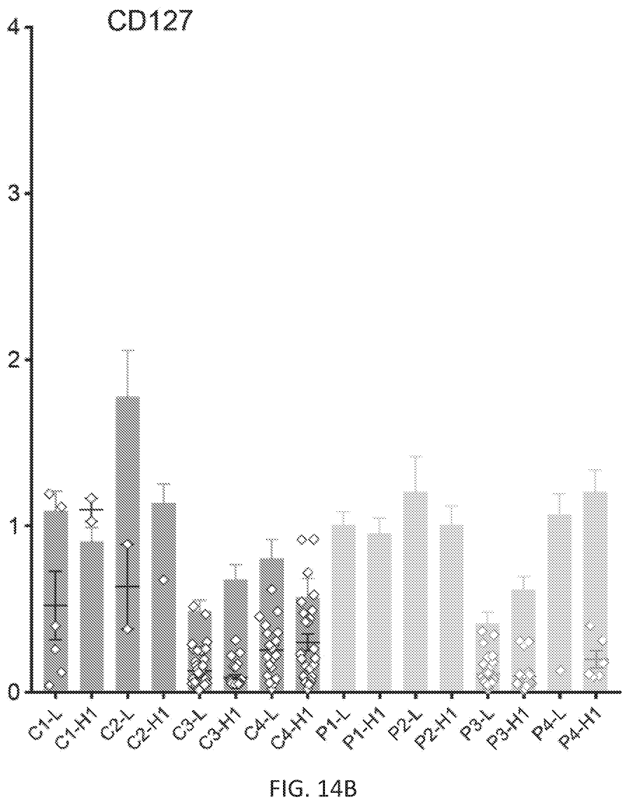

The single-cell sequencing pipeline¹⁹ has the advantage of linking phenotypic features with TCRα/β from individual cells. Thus, we examined phenotypic features associated with in vivo expanded clonotypes, with particular focus on eTRAJ24L⁺ cells. Phenotypes of expanded clones compared to unexpanded clones significantly (P<0.0001) differed for TBX21 (255/296, 86.1% vs 416/2282, 18.2%), IFNγ (108/296, 36.5% vs 53/2282, 2.3%), and PRF1 (149/296, 50.3% vs 105/2282, 4.6%) among 25 tested transcriptional factors and cytokines (FIGS. 7A, 7B and 14A). Consistent with the significantly higher numbers and/or more isolates of expanded clones from six patients (P3, P4, P7-9, and P12) and three vaccinated controls (C3, C4, and C11) (FIG. 4A), TBX21 (encoding T-bet) and PRF1 (encoding perforin) were found more frequently in these nine donors (FIG. 13B). The skewed expression pattern in expanded clones among donors was specific for TBX21 and PRF1 (FIG. 13B), supporting a previously proposed immune mechanism in which the in vivo expansion of perforin-expressing CD4⁺ T clones with cytotoxic potential was dependent on T-bet³⁸.

This phenotypic linkage was particularly informative in the context of T1N-associated in vivo proliferation and clonal expansion, because comparing 21 eTRAJ24L⁺ cells only found in patients to 18 unexpanded TRAJ24L⁺ cells found in many DQ6⁺ individuals showed an even more dramatically different frequency for TBX21 (95.2% vs 11.1%) and PRF1 (81.0% versus 0%) (FIGS. 7C, 7D). In line with differentiation into Tₕ₁ effectors, we found a slightly lower GATA3 frequency in eTRAJ24⁺ cells (42.9%) than the overall average (1415/2578, 54.9%), and extremely low detection of RORC(0), FOXP3 (0), and BCL6 (2) mRNAs. Consistent with lack of FOXP3, expression levels of other regulatory T (T_{reg}) cell markers like CD25 and CD127 on the surface of expanded clones including the 21 eTRAJ24⁺ cells, showed no difference from the levels on the tetramer^{neg}/CD4⁺ population (FIG. 14). Indeed, the solo isolate expressing an identical clonotype as in TCR27 from C12 (FIG. 5C) had 0 phenotypic transcripts (FIG. 7C). These phenotypic findings further confirmed the sequencing results indicating in vivo expansion of eTRAJ24L⁺ cells. Notably, although a TRAJ24F⁺ clone from C3 also expanded in vivo into effectors with Tₕ₁ phenotype (TBX21 frequency=100% vs 14.3% in unexpanded TRAJ24F⁺ cells), these 22 cells tended to express PRF1 (40.9% vs 81.0%) and TGF-β(18.2% vs 71.4%) less frequently, but express IFNγ (77.3% vs 9.5%) more frequently than the 21 eTRAJ24L⁺ cells, implying different functions between non-risk-carrying clones in controls and risk-carrying clones in patients (FIG. 7D). Collectively, these data suggested that DQ6-restricted eTRAJ24L⁺ cells in T1N patients have gone through T1N-related in vivo expansion and acquired cytotoxic potential, likely after recognition of epitopes in HCRT₅₆₋₆₉ or HCRT₈₇₋₁₀₀.

### DISCUSSION

In this study, we focus on using in vivo clonal expansion together with other genetic and molecular signatures to demonstrate the existence of autoreactive CD4⁺ T cells and link their expansion to a likely autoantigen and more importantly to T1N. Tetramer staining offers an approach to isolate cells of interest, which must then be further assessed to pinpoint autoreactive T cells among tetramer⁺ candidates. In particular, the discordance between tetramer binding and TCR signaling, observed here in self-antigen binding T cells and previously in foreign-antigen binding T cells²⁰-2, requires direct measurement of the signaling capacity of candidate autoreactive TCR.

The single-cell analysis we used uniquely allows pairing of TCRα/β and linkage with informative phenotypic characteristics. Of most importance, this approach allowed us to identify DQ6-HCRT_{peptide} tetramer⁺/CD4⁺ T cell clones expressing TRBV29-1_J2-5/TRAV6_J24 genes from among thousands of accessible single cells with paired α/β CDR3 sequences, from DQ6⁺ individuals with/without T1N. This is already remarkable given the huge diversity in TCR clonotytpes²⁰ and the likely involvement of polyclonal TCRs in T1N¹⁵. More strikingly, sequence/phenotype-determined in vivo expansion of eTRAJ24L clonotypes that carry the T1N risk allele encoding the J24L variant are exclusively observed in T1N patients. The relevance of clonal expansion to the disease-relatedness of these cells is also reflected by the lack of expansion and the absence of Tₕ₁ phenotypic markers in the solo isolate sharing identical α/β CDR3s with TCR27 from control PBMCs. However, the presence of this clonotype may constitute a significant disease risk element. Furthermore, all eTRAJ24L clonotypes including TCR27 can bind homologous NHAAGILTL(M) (SEQ ID NO:34) epitopes from the C-termini of the two neurotransmitters, HCRT1 and HCRT2. The Jurkat-reporter transfectant expressing TCR27 shows a significant signaling response to HCRT₈₇₋₉₇-NH2 (SGNHAAGILTM-NH2, SEQ ID NO:37), a peptide that is physiologically available for DQ6-restricted presentation in all DQ6⁺ individuals prior to disease onset.

HLA class II expression in the central nervous system (CNS) is restricted to microglia and certain neural progenitors³⁹. Thus, the presentation of NHAAGILTL(M) (SEQ ID NO:34) epitopes to naïve CD4⁺ T cells may occur through presentation by neuron-associated microglia expressing DQ6. CD4⁺ Tₕ₁ effectors could then activate microglia to secrete neurotoxic factors or exert their own cytotoxic potential, as suggested in our phenotypic analysis, to destroy microglia. Either process would be detrimental to the associated neurons⁴⁰. In addition, fragments of HCRT proteins that include the epitopes may leave the CNS, and prime Tₕ, effectors outside of the brain, similar to a recent murine model in which insulin peptides released by pancreatic β-cells initiate diabetic T cell responses at distant lymph nodes⁴¹. Our finding of eTRAJ24L clonotypes in T1N patient PBMCs suggest the presence and persistence of brain tissue-reactive TCR clonotypes in the circulation, reminiscent of recent evidence that gluten-specific TCR clonotypes persist in blood and overlap with clonotypes in gut biopsies from celiac patients⁴².

Many expanded clonotypes including eTRAJ24L cross-bind to more than one DQ6-HCRT_{peptide} tetramer. TCRs can recognize class II bound to peptides that share certain homology but differ at some TCR-facing residues¹⁷, likely due to the ability of a TCR to use various modes of ligand interaction⁴³. Our observation either reflects a general characteristic of autoreactive T cells or implies a unique promiscuous feature of DQ6-reactive TCRs. The former is consistent with the finding that many memory T cells express cross-reactive TCRs¹⁷. The latter is supported by the NetMHCIIpan²⁶ prediction that alanine is preferred at all core residues of DQ6-binding peptides that lack side chains to increase TCR specificity. Indeed, all five regions of prepro-HCRT that generate DQ6-binding peptides contain multiple alanine residues. The cross-reactive potential also provides molecular insight into how viral proteins could have promoted T1N during the 2009 flu pandemic^{33,34}, for example by mimicking HCRT epitopes.

Expanded clonotypes other than eTRAJ24L (including the TRAJ24F-expressing clone found in a control) with tetramer cross-reactivity or specificity from our dataset may also mediate ongoing immune responses, either associated with (including regulatory function) or irrelevant to T1N. Because tetramer⁺ cells do not necessarily respond to the HLA-peptide complex, cross-tetramer binding also does not ensure signaling to both epitopes, as suggested by the failure of TCR27-mediated signaling in response to HCRT₁₋₁₃. The physiological significance of thymic selection for many self-binding, but non-autoreactive, cells may be to maintain self-tolerance, for example by selectively sequestrating HCRT peptides, which would otherwise trigger signaling competent T cells for autoimmunity. Also notable is that TRAJ24L as an inherited risk allele may be only present in a subgroup of DQ6⁺ individuals or T1N cases. Therefore, polyclonal TCRs or factors other than TCRs may be involved in T1N developing in different individuals. Nonetheless, our discovery of eTRAJ24L clonotypes provides novel biomarkers of disease risk/diagnosis or potential therapeutic targets in early disease.

The two homologous epitopes at the C-termini of HCRT1 (NHAAGILTL, (SEQ ID NO:33) and HCRT2 (NHAAGILTM, SEQ ID NO:34) may make different contributions to autoimmunity rather than redundantly boosting the response. Indeed, HCRT1 and HCRT2 proteins differ in stability and binding affinities to HCRTR1 and HCRTR2 receptors²⁸, with possible consequences for tolerance. Our findings thus raise the next set of mechanistic questions, while uncovering autoimmune effectors and targets in T1N.

### Example 2

### Materials and Methods

Construction of Recombinant HLA-DQ6 in Complex with HCRT Peptides

A stable Drosophila Schneider 2 (S2) insect cell line secreting soluble DQ6 proteins was previously constructed⁴⁴. In this construct, the class II-associated invariant chain peptide, CLIP₈₇₋₁₀₁ (aa: PVSKMRMATPLLMQA, SEQ ID NO:38), is covalently linked to the p chain of DQ6 a/β heterodimers. The DQ6 construct includes the extracellular portion of HLA-DQA1*01:02 followed by a 3C protease cleavage site and leucine zipper-Fos sequence and the extracellular portion of HLA-DQAB1*06:02, preceded by the peptide sequence and a thrombin-cleavable linker sequence (GGGGSLVPRGSGGGG, SEQ ID NO:39), and followed by a 3C protease cleavage site and leucine zipper-Jun sequence. Similarly, we constructed five S2 cell lines expressing soluble DQ6-HCRT₁₋₁₃ (aa: MNLPSTKVSWAAV, SEQ ID NO:40), DQ6-HCRT₂₅₋₃₇ (aa: ALLSSGAAAQPLP, SEQ ID NO:41), DQ6-HCRT₅₆₋₆₉ (aa: AGNHAAGILTLGKR, SEQ ID NO:42), DQ6-HCRT₈₇₋₁₀₀ (aa: SGNHAAGILTMGRR, SEQ ID NO:43) and DQ6-EBV₄₈₆-4₉₉ (aa: RALLARSHVERTTD, SEQ ID NO:44), respectively. Briefly, two plasmids (encoding α and β chains of DQ6) were used for the expression of each DQ6-peptide complex in S2 cells. The a chain-encoding plasmid is shared by all constructs, and the p chain-encoding plasmids were modified via polymerase chain reactions (PCR) and sub-cloning to swap the nucleotide sequence encoding corresponding peptides that were covalently tethered to the N-terminus of DQ6p. S2 cells were co-transfected following the user guide for Drosophila S2 cells (Invitrogen, Thermo Fisher Scientific) with α- and β-encoding plasmids as well as a third plasmid carrying the neomycin (geneticin)-resistance gene, at a ratio of 20:20:1. Geneticin (G418)-resistant S2 transfectants were recovered after 2-3 weeks of culturing in Schneider Drosophila medium with 10% heat inactivated fetal bovine serum (HI FBS), 2 mM glutamine and 1.5 mg/ml G418 (all from Thermo Fisher Scientific). Stable cell lines were established after another 2-3 weeks of culturing and selection under G418.

### Expression and Purification of Soluble HLA Proteins

Stable S2 cell lines secreting soluble HLA proteins (e.g., a DQ6-HCRT_{peptide} complex or DM⁴⁴) were initially cultured in the complete Schneider medium and gradually adapted to S2 serum-free medium (Thermo Fisher Scientific) before the induction of protein expression using 1 mM copper sulfate. After 1-week induction, bacteriostatic protease inhibitors, such as 1 mM phenylmethane sulfonyl fluoride (PMSF), 1 mM ethylenediaminetetraacetic acid (EDTA), and 0.02% sodium azide (NaN₃), were added to the S2 culture, which was then centrifuged to collect supernatants containing soluble HLA proteins. The 0.22 micron membrane-filtered supernatant was then applied onto a column for the purification of target proteins by affinity chromatography. A customized anti-DQ column containing SPV-L3 Ab⁴⁴ was used to purify DQ6 and a column composed of M2 (anti-FLAG tag) resins (Sigma) was used to purify DM. Affinity-purified proteins were further concentrated and isolated from aggregates or degraded material by size exclusion chromatography, using either Superdex increase 200 10/300 GL or HiLoad 16/60 Superdex 200 gel filtration columns (GE Healthcare). Fractions were eluted with TBS buffer (e.g., 20 mM Tris-Cl, 150 mM NaCl, pH 7.4) and the ones containing monomeric forms of each protein were pooled. Protein purity was confirmed using Coomassie and western blotting analyses, and protein functionality was validated in the peptide-binding assay, as described below.

### Peptide Competition Assay

The ability of a peptide to inhibit the interaction of DQ6 and a reference binding peptide at steady state was used to estimate the relative DQ6 binding capacity of test peptides. We used biotinylated EBV₄₈₆₋₅₀₀ (aa: biotin-GGGRALLARSHVERTTDE (SEQ ID NO:45), synthesized by Genscript), a DQ6-binding peptide (epitope underlined) derived from Epstein-Barr virus nuclear antigen²⁵, as our reference peptide. Non-biotinylated test peptides included thirty 15-mer overlapping peptides derived from prepo-HCRT (Table 1, by Genscript) and the positive control peptide EBV₄₈₆₋₅₀₀. The DQ6-CLIP₈₇₋₁₀₁ construct contains a thrombin cleavage site in between CLIP₈₇₋₁₀₁ and DQ63. To cleave the covalent linker and enable replacement of CLIP₈₇₋₁₀₁ by high affinity DQ6 binders, soluble DQ6-CLIP₈₇₋₁₀₁ at a concentration of 3 μM was incubated with 0.002 U/μl thrombin enzyme (Novagen, EMD Millipore) for 2 h at room temperature (RT) prior to peptide loading experiments. To test DQ6 binding capacity, a non-biotinylated peptide at 40 μM was mixed with 1 μM biotinylated EBV₄₈₆₋₅₀₀ and incubated with 25 nM thrombin-cleaved DQ6-CLIP₈₇₋₁₀₁. 100 nM soluble DM was added as a catalyst to increase the peptide exchange efficiency⁴⁴. The reaction was carried out under acidic conditions in 100 mM acetate buffer (acetic acid and sodium acetate, pH 4.6), 150 mM NaCl, 1% (w/v) BSA, 0.5% (v/v) IGEPAL CA-630 (Sigma), 0.1% (w/v) NaN₃, at 37° C. for 20 hours. After incubation, the peptide exchange reaction was stopped by the addition of two volumes of the neutralization buffer [100 mM Tris-Cl (pH 8.3), 150 mM NaCl, 1% (w/v) BSA, 0.5% (v/v) IGEPAL CA-630, 0.1% (w/v) NaN₃], and the mixture was transferred to an SPV-L3-coated 96-well plate and incubated at RT for 1 h. Time resolved fluorescence representing DQ6-associated biotinylated EBV₄₈₆₋₅₀₀ captured by SPV-L3 in each well was then quantified using the DELFIA Eu-N1 Streptavidin System (PerkinElmer).

### DQ6-HCRT Crystallization and Structure Determination

DQ6-HCRT₅₆₋₆₉ proteins purified from S2 culture were incubated with recombinant HRV 3C protease (3C^{pro}, Novagen, EMD Millipore) at 4° C. overnight to remove the leucine zipper at the C-termini of DQ6α/β heterodimers. 3C^{pro}-cleaved DQ6-HCRT₅₆₋₆₉ was further purified by anion exchange chromatography using HiTrap Q HP and finally by gel filtration using HiLoad 16/60 Superdex 200 (GE healthcare). For crystallization, DQ6-HCRT₅₆₋₆₉ was concentrated to 10 mg/ml in 20 mM Tris-Cl pH 7.5, 20 mM NaCl, 0.01% NaN₃. Thin elongated plates measuring approximately 300×100×20 μm were obtained after 5 days at room temperature by mixing 1 μl of protein with 1 μl of precipitant solution containing 16% PEG 8K, 0.1 M Mg acetate, and 0.1 M glycine pH 4.5. Crystals were flash-frozen by mixing 75% mother liquor (v/v) with 25% saturated sucrose. X-ray diffraction data were recorded at 100 K (λ=0.9793 Å) at the LRL-CAT 31-ID beamline Advanced Photon Source (APS) in Chicago. Images were processed using Mosflm version (7.1.1)⁴⁵ and scaled with SCALA⁴⁶. Initial phases were obtained by molecular replacement with Phaser⁴⁷ based on the DQA1*01:02/DQB1*06:02 α- and β-chains from PDB file 1UVQ²⁷. One strong molecular replacement solution was found with 1 molecule per asymmetric unit each of the α- and β-chains. The solution was confirmed by examination of composite omit maps. After one round of rigid body refinement, the hypocretin peptide was built manually and the whole model improved by cycles of manual building and refinement using COOT⁴⁸ and PHENIX REFINE⁴⁹ respectively. The overall geometry in the final structure is good, with 98.6% of residues in favored regions, 1.4% in allowed regions of the Ramachandran plot and no outliers. Data collection and refinement statistics are reported (Table 2). Residues 105-112 of the β-chain are missing in the electron density, likely due to disorder, and were not included in the structural model. Residues 56-68 of the DQ6-HCRT56-69 peptide sequence AGNHAAGILTLGK was built into clear electron density in the peptide binding cleft, but no electron density was observed for arginine 69 and the linker (GGGGSLVPRGSGGGG, SEQ ID NO:39) tethering the peptide to the N-terminus of the p chain. A monosaccharide of N-acetyl glucosamine was built at asparagine residues 81 and 121 of the a chain and a disaccharide at asparagine 19 of the p chain. One molecule of Tris was modeled into the electron density. Two amino acid side chains in the p chain were disordered (Arg 48 and Glu 59) and were refined with two alternative conformations. Structural biology software used in this project was curated by SGgrid⁵⁰. Structure figures were generated using the program PyMOL⁵¹.

### In Silico Analysis Using NetMHCIIPan

We used the MHC-II peptide binding prediction website, NetMHCIIpan 3.2²⁶, to evaluate potential DQ6-binding core epitopes within the prepro-HCRT sequence. The resultant in silico predictions of binding rank for HCRT-derived peptides at various lengths are reported (FIG. 8). The peptides containing strong predicted core registers were analyzed in experimental binding assays. The NetMHCIIpan 3.2 motif viewer displays binding motifs and predicts that DQ6 (DQA1*0102/DQB1*0602) prefers alanine over all other residues at each of the 9 anchor positions of a potential binding peptides, with small residues such as serine, glycine, and threonine also preferred at most of the positions.

### DQ6-HCRT Modeling and Structural Analysis

Models for HCRT-derived peptides bound to DQ6 were developed using the DQ6-HCRT₅₆₋₆₉ structure. HCRT-derived peptides shown to bind to DQ6 by competition binding studies were docked onto the DQ6-HCRT₅₆₋₆₉ structure using the 9-aa core epitope defined by NetMHCIIpan for alignment. Peptide side chain rotamers and if necessary DQ side chain rotamers were adjusted using Pymol⁵¹ to accommodate the sequence changes without steric clashes; adjustment of peptide or DQ6 main chain conformation was not required.

### Human Subiects and Peripheral Blood Samples

All donors in this study are HLA-DQB1*06:02⁺. Narcoleptic patients with cataplexy met the criteria for International Classification of Sleep Disorders 3 (ICSD3) for type 1 narcolepsys⁵². The controls are either unrelated or influenza-vaccinated subjects. Influenza vaccines included Pandemrix (an AS03-adjuvanted 2009 H1N1 influenza vaccine formulation, GSK) or a seasonal trivalent inactivated influenza vaccine (TIV, Fluzone, NDC 49281-705-55, 2012-2013 formula, Sanofi Pasteur). PBMCs were received from the Stanford Center for Sleep Sciences and Medicine. Written consent was obtained in all cases under a Stanford Institutional Review Board approved protocol, following the guidelines for human subjects' research under U.S. Department of Health and Human Services human subjects regulations (45 CFR Part 46).

### Tetramer Synthesis

Customized DQ6-peptide tetramers were all synthesized by NIH Tetramer Core Facility at Emory University using monomers that were secreted from a mammalian cell expression system. These recombinant DQ6-peptide monomers including DQ6-HCRT₁₋₁₃, DQ6-HCRT₂₅₋₃₇, DQ6-HCRT₅₆₋₆₉, DQ6-HCRT₈₇₋₁₀₀, and DQ6-EBV₄₈₆₋₄₉₉ used identical constructs as mentioned above in the S2 expression system. In each tetramer, peptides are covalently tethered to the N-terminus of DQ63 in order to maintain the peptide specificity.

### In Vitro Culturing of CD4⁺ T Cells

To test for the presence of DQ6-HCRT_{peptide} tetramer⁺/CD4⁺ T cells, a peptide-loaded antigen presenting cell (APC) line T2DQ6 (fixed to limit APC proliferation) was co-cultured with T cells for DQ6-restricted antigen stimulation. T2DQ6 was constructed by stable transfection of DQ6 (DQA1*01:02/DQB1*06:02) into T2, a class II deficient TxB hybrid cell⁴⁴. T2DQ6 cells were maintained in IMDM, GlutaMAX supplemented media (Thermo Fisher Scientific) with 10% HI FBS, 1% penicillin/streptomycin (P/S) and 1 mg/ml G418 (to maintain selective pressure on DQ6 transfectants). To load antigen, T2DQ6 cells were pulsed with peptides (i.e., EBV₄₈₆₋₅₀₀, HCRT₅₆₋₆₉, HCRT₈₇₋₁₀₀, synthesized by Genscript) at 1 μM final concentration and incubated for 6 hours at 37° C. After peptide loading, 10 million T2DQ6 cells were washed with phosphate buffered saline (PBS) and then fixed by incubating with 0.025% glutaraldehyde in 2 ml PBS at RT for 30 seconds. After the addition of another 2 ml PBS, the cells were incubated for another 10 minutes at RT. Fixed T2DQ6 was washed twice with PBS and once with complete RPMI (RPMI 1640 medium supplemented with 10% HI human AB serum, 2 mM glutamine and 1% PS) before mixing with CD4⁺ T cells. Human PBMCs frozen in NUNC tubes were thawed quickly at 37° C. and added slowly to 10 ml warm complete RPMI. PBMCs were pelleted and resuspended in cold buffer for CD4⁺ T cell isolation. Cells were isolated from the PBMCs by negative selection, using the CD4⁺ cell isolation kit, according to the manufacturer's instructions (Miltenyi Biotec). Isolated CD4⁺ T cells were resuspended at 1×10⁶ cells/ml in warm complete RPMI and rested for at least 1 hour before mixing with fixed T2DQ6 cells that were also resuspended at 1×10⁶ cells/ml in warm complete RPMI. A mixture of 1:1 volume ratio of CD4⁺ T cells and T2DQ6 cells in the presence of recombinant human IL-7 at final concentration of 2.5 ng/ml was aliquoted onto a 96-well plate and incubated at 37° C. for 6 days. On day 6 and day 9, 100 μl of the spent medium was removed from each well and replaced with fresh complete RPMI containing recombinant IL-7 at 2.5 ng/ml and IL-2 at 40 U/ml final concentrations. On day 12, a second round of antigen stimulation was performed similarly, using fixed T2DQ6 cells loaded with the corresponding peptides.

### Analysis of Tetramer*/CD4⁺ Cells in the In Vitro T Cell Culture

Sufficient cells from the co-culture were collected, washed with complete RPMI, and resuspended in 5 ml complete RPMI at RT prior to Ficoll gradient separation. Cells above the Ficoll media were washed and resuspended at 10×10⁶ cells/ml in complete RPMI for blocking. After 10 min, tetramers were added to a final concentration of 30 μg/ml and staining was performed for 30 min at 37° C. in the dark, followed by another 15 min incubation at RT with the addition of Alexa fluor 488 anti-human CD4 and PerCP-Cy5.5 anti-human CD19 Abs (BioLegend, to separate T cells from the TxB hybrid T2 cells). Cells were then washed twice with chilled PBS+10% HI FBS and resuspend in 200 l PBS+10% HI FBS for flow cytometric analysis. Live/dead dyes such as propidium iodide (PI, Thermo Fisher Scientific) or Via Probe (7-AAD, BD Biosciences) were added to each sample before acquisition on a flow cytometer. Cytometers included FACSCallibur, LSR II, and FACSAria II (BD Biosciences).

Single Cell Index Sorting (iFACS) of Tetramer*/CD4⁺ Cells

Frozen PBMCs were received as randomized pairs each composed of one patient sample with one control sample for a blinded study. The Mellins laboratory performed two sets of independent experiments using PBMCs from 12 patient/control pairs of DQ6⁺ donors. In Set A, cells of control (C) or patient (P) subjects 1-4 were stained with DQ6-HCRT₁₋₁₃ or DQ6-HCRT₅₆₋₆₉ tetramer. In Set B, cells of C or P 5-12 were stained with DQ6-HCRT₁₋₁₃ or DQ6-HCRT₈₇₋₁₀₀ tetramer and selected samples (C7, 8, 11, 12, and P7-12) were stained with HCRT₂₅₋₃₇ tetramer (FIG. 4A). Paired PBMC samples were thawed and used for CD4 T cell isolation, as described above. Cell viability was maintained by minimizing the exposure of primary CD4⁺ T cells to temperatures higher than 4° C. 3-5 million CD4⁺ T cells were labeled with LIVE/DEAD cell stains (Life Technologies, Thermo Fisher Scientific) in PBS on ice for 30 min, and then washed and incubated at a density of 10×10⁶ cells/ml in complete RPMI with one of the following tetramers: DQ6-HCRT₁₋₁₃, DQ6-HCRT₂₅₋₃₇, DQ6-HCRT₅₆₋₆₉, DQ6-HCRT₈₇₋₁₀₀ at a final concentration of 50 μg/ml at 37° C. for 15 min. After the addition of anti-CD4 and anti-CD19 Abs, staining was performed on ice for another 3 h. In Set A, anti-CD127 and anti-CD25 Abs (BioLegend), in addition to anti-CD4 and anti-CD19 Abs, were used to evaluate the subsets of tetramer⁺/CD4⁺ T cells in the FACS experiment. Cell samples were then washed in PBS+1% bovine serum albumin (BSA) and applied on a FACSARIA II cell sorter in the Stanford Shared FACS Facility for the single cell fluorescence-activated cell sorting (FACS). Up to 96 tetramer⁺/CD4⁺ cells per sample were individually sorted into a 96-well PCR plate (Eppendorf) with each well containing 10 μl of 1× OneStep RT-PCR buffer (QIAGEN). The index feature associated with the single cell FACS (iFACS) allowed recording of fluorescence intensity (FI) parameters of each sorted cell.

### Index Analysis

Data including index Fl values at each channel for sorted single cells were exported from the FACSDIVA software. To determine the tetramer binding rank of sorted clones within a specific subject-tetramer category, index Fl at the tetramer channel normalized by forward scatter (FSC) intensity of each single tetramer⁺/CD4⁺ cell was compared with MFI of the tetramer^{neg}/CD4⁺ cell population that was normalized by mean FSC intensity.

### Deep Sequencing of TCR and Phenotypic Transcripts in Single T Cells

Single cell mRNA sequencing was performed after three rounds of nested PCR amplification of TCR and phenotypic transcripts as described¹⁹ with some optimization. Briefly, OneStep RT-PCR (following QIAGEN manual) using single cells as the template in the same 96-well PCR plate into which tetramer⁺/CD4⁺ cells were sorted was initiated on the same day when iFACS was accomplished. The annealing temperature was set to 58° C. (used for all three rounds of PCR reactions). This first round of 15 μl multiplex PCR amplified 240-300 base pairs (bps) mRNA sequences of target TCR and phenotypic transcripts by a set of primers recognizing 38 TCRα genes, 36 TCR3 genes, and 25 selected phenotyping marker genes. TCR amplicons cover the V(D)J regions including CDR3 sequences. Two slightly different pairs of specific primers were applied to amplify IFN-γ transcripts in Set A versus Set B donor samples. TCR and phenotypic amplicons from the same cell were then further amplified in separate 96-well PCR plates in a second round of multiplex PCR (15 μl) using 1 μl of the RT-PCR products as the template and HotStarTaq enzyme (QIAGEN) as the DNA polymerase. The second round PCR amplicons (200-250 bps) in selected wells were validated by gel electrophoresis. In the third round of amplification, 1 μl aliquot of the second PCR products (TCR or phenotyping, separately) was used as a template in 15 μl PCR reaction, which incorporates Illumina paired-end (PE) sequences and a unique pair of barcodes with amplicons in each well. The third round PCR amplicons with a length of 350-380 bps from each well were pooled at equal proportion by volume and purified from 2% agarose gel using Qiaquick gel extraction kit (QIAGEN). The incorporated PE sequences enabled deep sequencing on the Illumina Miseq platform (Human Immune Monitoring Center at Stanford University), whereas barcodes allowed deconvolution of deep sequencing data.

### Sequencing Data Analysis

The previously described VDJFasta algorithm¹⁹ was used to de-multiplex raw sequencing data and assign each sequence read to a particular well in each PCR plate according to unique plate-row-column barcodes. The average read number per well was 6,091±4,556. Reads with at least 95% sequence homology were assumed to derive from a consensus sequence of the same TCR. A consensus TCRβ sequence with over 80% reads in a well (BetaConfi>80%) was assigned to the cell. The top one consensus TCRα sequence with over 30% reads in a well (AlphaConfi>30%) was assigned to the cell as the dominant TCRα; whereas a second consensus TCRα sequence with over 10% reads in the same well (altalAphaConfi>10%), if any, was assigned to the cell as the alternative TCRα. For phenotyping markers of the cell, the total reads containing at least 95% sequence homology to a transcription factor or cytokine gene were scored. Both Illumina MiSeq deep sequencing and data analysis were performed at the Human Immune Monitoring Center at Stanford University.

### GLIPH Analysis

The recently developed GLIPH algorithm²⁰ was used to cluster TCRs with a high probability of sharing specificity due to the similarity among their CDR3P sequences. Based on the extent of sequence similarity, three types of conserved motifs were classified: global, local, or single motifs, respectively. Members in a motif group with significant enrichment of common Vβ genes and clonal expansion were summarized to reveal expanded TCR clones isolated in more than one DQ6⁺ donor.

### Construction of Jurkat Cell Lines Expressing Candidate TCRs

To encode the entire α/β chains of a candidate TCR, the nucleotide sequences for CDR3a and CDR3P were incorporated in frame with the corresponding V(D)J genes (IMGT/V-QUEST³⁷). TCRα/β genes were then synthesized and cloned into a plasmid pEF1α-TCRA_2A_TCRB_IRES-AcGFP1 (by GenScript). Each reconstructed plasmid uses a mammalian promoter, EF1a to direct co-expression of α and β chains of one candidate TCR. TCRA and TCRB are separated by the 2A self-cleaving peptide sequence. For selection purposes, the plasmid also encodes the green fluorescence protein (GFP) and neomycin resistance gene, with expression driven by separate promoters. TCR-encoding plasmids were then transfected into the TCRα/β deficient Jurkat cell line (J76-NFATRE-luc)²⁰ by nucleofection following the Amaxa Optimized protocol (Lonza). Transfectants were selected by G418 at a concentration of 1 mg/ml and recovered after 3-4 weeks of culturing in the RPMI medium supplemented with 10% HI FBS, 2 mM glutamine, and 1% PS. To avoid the heterogeneity in TCR expression observed in a polyclonal cell line, TCRαβ⁺/GFP⁺/CD3⁺ cell transfectants were individually sorted by single cell FACS, to expand clonal cell lines originating from single cell transfectants. These clonal lines were then co-stained with PE anti-TCRα/β Abs (BD Biosciences) and APC DQ6-HCRT tetramers to confirm the expression of TCRs and validate their binding to tetramers using flow cytometric analysis.

### T Cell Activation Assay Using a Luciferase Reporter

The TCR transfectants of J76-NFATRE-luc cells expresses the NFAT-RE (response element)-luciferase reporter gene allowing the conversion of T cell activation signaling to luciferase activity²⁰. 1×10⁵ K562-DQ6 cells (an artificial APC line expressing the only HLA allele: DQA1*01:02/DQB1*06:02)²⁰ and 1×10⁵ single clonal expanded TCR transfectants of J76-NFATRE-luc cells were mixed in 100 μl RPMI medium supplemented with 10% HI FBS, 2 mM glutamine, and 1% PS. The co-culture was incubated with various stimuli at 37° C. for 1 day before quantification of the luciferase activity. Stimuli included 10-50 mM of one HCRT peptide (i.e., HCRT₁₋₁₃, HCRT₂₅₋₃₇, HCRT₈₇₋₁₀₀, and HCRT₈₇₋₉₇-NH2, synthesized by Genscript), or 1 μg/ml anti-CD3+1 μg/ml anti-CD28 Abs (BioLegend) as the positive control, or an equal volume of PBS as the negative control. After incubation, 50 μl of co-culture was mixed with 50 μl of luciferase substrate provided in the Nano-Glo Luciferase Assay System (Promega). The mixture was then transferred to a flat-bottom white 96-well plate (Costar) for a measurement of chemiluminescence using a plate reader.

### Phenotypic Analysis

The number of sequencing reads of a phenotyping marker in a well is dependent on the number of amplicons resulting from three rounds of nested PCR reactions. This number does not necessarily reflect the transcriptional level of the marker per cell, as PCR may bias for transcripts whose amplification occurs relatively efficient at the conditions described above. To eliminate any PCR bias in this semi-quantitative sequencing approach, we assigned 1 or 0 to indicate the presence or absence of reads of a phenotypic transcript in a well without weighing its actual reads. Comparison between patient and control samples was then performed for the overall dataset or TCR clones clustered according to different schemes (FIG. 13A). Numbers of cells from different donors with various tetramer binding specificities that express each phenotypic transcript were also compared (FIG. 13B).

### Statistical Analysis

Most of the statistical analyses performed in this study compared a single variable between two groups of samples, such as patients versus controls, one tetramer binding specificity versus another, or one TCR cluster versus another. In these situations, a two-tailed Student's t-test was used to determine the statistical significance of the difference, with significance defined as P<0.05. Welch's t-test was used if the two groups of samples had unequal variances or unequal sample sizes. The paired samples t-test was used if there were matched pairs of samples in the two groups. The multiple t-test was used when more than two groups were compared. All statistics were performed with GraphPad Prism, using the built-in analysis.

TABLE 1
15-mer overlapping peptides derived from prepro-
HCRT (offset by 4 aa).

| ID | Name | Sequence |
|---|---|---|
| 1 | Hcrt1-15 | MNLPSTKVSWAAVTL (SEQ ID NO: 1) |
| 2 | Hcrt5-19 | STKVSWAAVTLLLLL (SEQ ID NO: 2) |
| 3 | Hcrt9-23 | SWAAVTLLLLLLLLP (SEQ ID NO: 3) |
| 4 | Hcrt13-27 | VTLLLLLLLLPPALL (SEQ ID NO: 4) |
| 5 | Hcrt17-31 | LLLLLLPPALLSSGA (SEQ ID NO: 5) |
| 6 | Hcrt21-35 | LLPPALLSSGAAAQP (SEQ ID NO: 6) |
| 7 | Hcrt25-39 | ALLSSGAAAQPLPDC (SEQ ID NO: 7) |
| 8 | Hcrt29-43 | SGAAAQPLPDCCRQK (SEQ ID NO: 8) |
| 9 | Hcrt33-47 | AQPLPDCCRQKTCSC (SEQ ID NO: 9) |
| 10 | Hcrt37-51 | PDCCRQKTCSCRLYE (SEQ ID NO: 10) |
| 11 | Hcrt41-55 | RQKTCSCRLYELLHG (SEQ ID NO: 11) |
| 12 | Hcrt45-59 | CSCRLYELLHGAGNH (SEQ ID NO: 12) |
| 13 | Hcrt49-63 | LYELLHGAGNHAAGI (SEQ ID NO: 13) |
| 14 | Hcrt53-67 | LHGAGNHAAGILTLG (SEQ ID NO: 14) |
| 15 | Hcrt57-71 | GNHAAGILTLGKRRS (SEQ ID NO: 15) |
| 16 | Hcrt61-75 | AGILTLGKRRSGPPG (SEQ ID NO: 16) |
| 17 | Hcrt65-79 | TLGKRRSGPPGLQGR (SEQ ID NO: 17) |
| 18 | Hcrt69-83 | RRSGPPGLQGRLQRL (SEQ ID NO: 18) |
| 19 | Hcrt73-87 | PPGLQGRLQRLLQAS (SEQ ID NO: 19) |
| 20 | Hcrt77-91 | QGRLQRLLQASGNHA (SEQ ID NO: 20) |
| 21 | Hcrt81-95 | QRLLQASGNHAAGIL (SEQ ID NO: 21) |
| 22 | Hcrt85-99 | QASGNHAAGILTMGR (SEQ ID NO: 22) |
| 23 | Hcrt89-103 | NHAAGILTMGRRAGA (SEQ ID NO: 23) |
| 24 | Hcrt93-107 | GILTMGRRAGAEPAP (SEQ ID NO: 24) |
| 25 | Hcrt97-111 | MGRRAGAEPAPRPCL (SEQ ID NO: 25) |
| 26 | Hcrt101-115 | AGAEPAPRPCLGRRC (SEQ ID NO: 26) |
| 27 | Hcrt105-119 | PAPRPCLGRRCSAPA (SEQ ID NO: 27) |
| 28 | Hcrt109-123 | PCLGRRCSAPAAASV (SEQ ID NO: 28) |
| 29 | Hcrt113-127 | RRCSAPAAASVAPGG (SEQ ID NO: 29) |
| 30 | Hcrt117-131 | APAAASVAPGGQSGI (SEQ ID NO: 30) |

TABLE 2
Data collection and refinement for
crystallization of DQ6-HCRT₅₆₋₆₉.

| | DQ6-HCRT₅₆₋₆₉ |
|---|---|
| Data collection | |
| Space group | C 1 2 1 |
| Cell dimensions | |
| a, b, c (Å) | 63.67, 89.60, 90.93 |
| α, β, γ (°) | 90, 99.52, 90 |
| Resolution (Å) | 42.21-2.0 (2.07-2.0)* |
| R_{merge} | 0.12 (0.95) |
| CC_{1/2} | 0.99 (0.82) |
| \|Iσ\| | 5.68 (1.22) |
| Completeness (%) | 99.5 (98.9) |
| Redundancy | 3.8 (3.5) |
| Refinement | |
| Resolution (Å) | 42.21-2.0 |
| No. reflections | 33,905 (3356) |
| R_{work} | 22.2 (38.3) |
| R_{free} | 25.6 (39.1) |
| No. atoms | |
| Protein | 3036 |
| Ligand/ion | 64 |
| Water | 272 |
| B-factors | |
| Protein | 39.2 |
| Peptide | 35.7 |
| Ligand/ion | 82.5 |
| Water | 42 |
| R.m.s. deviations | |
| Bond lengths (Å) | 0.014 |
| Bond angles (°) | 1.57 |

*Values in parentheses are for highest-resolution shell.

### REFERENCES

1 Longstreth, W. T., Jr., Koepsell, T. D., Ton, T. G., Hendrickson, A. F. & van Belle, G. The epidemiology of narcolepsy. Sleep 30, 13-26 (2007).
2 Peyron, C. et al. A mutation in a case of early onset narcolepsy and a generalized absence of hypocretin peptides in human narcoleptic brains. Nat Med 6, 991-997, doe:10.1038/79690 (2000).
3 Matsuki, K. et al. DQ (rather than DR) gene marks susceptibility to narcolepsy. Lancet 339, 1052 (1992).
4 Tafti, M. et al. DQB1 locus alone explains most of the risk and protection in narcolepsy with cataplexy in Europe. Sleep 37, 19-25, doi:10.5665/sleep.3300 (2014).
5 Hallmayer, J. et al. Narcolepsy is strongly associated with the T-cell receptor alpha locus. Nat Genet 41, 708-711, doi:10.1038/ng.372 (2009).
6 Han, F. et al. Genome wide analysis of narcolepsy in China implicates novel immune loci and reveals changes in association prior to versus after the 2009 H1N1 influenza pandemic. PLoS Genet 9, e1003880, doi:10.1371/journal.pgen.1003880 (2013).
7 Kornum, B. R. et al. Common variants in P2RY11 are associated with narcolepsy. Nat Genet 43, 66-71, doi:10.1038/ng.734 (2011).
8 Faraco, J. et al. ImmunoChip study implicates antigen presentation to T cells in narcolepsy. PLoS Genet 9, e1003270, doi:10.1371/journal.pgen.1003270 (2013).
9 Cvetkovic-Lopes, V. et al. Elevated Tribbles homolog 2-specific antibody levels in narcolepsy patients. J Clin Invest 120, 713-719, doi:10.1172/JCl41366 (2010).
10 Saariaho, A. H. et al. Autoantibodies against ganglioside GM3 are associated with narcolepsy-cataplexy developing after Pandemrix vaccination against 2009 pandemic H1N1 type influenza virus. J Autoimmun 63, 68-75, doi:10.1016/j.jaut.2015.07.006 (2015).
11 Tanaka, S., Honda, Y., Inoue, Y. & Honda, M. Detection of autoantibodies against hypocretin, hcrtrl, and hcrtr2 in narcolepsy: anti-Hcrt system antibody in narcolepsy. Sleep 29, 633-638 (2006).
12 Ahmed, S. S. et al. Antibodies to influenza nucleoprotein cross-react with human hypocretin receptor 2. Sci Transl Med 7, 294ra10⁵, doi:10.1126/scitranslmed.aab2354 (2015).
13 Luo, G. et al. Absence of anti-hypocretin receptor 2 autoantibodies in post pandemrix narcolepsy cases. PLoS One 12, e0187305, doi:10.1371/journal.pone.0187305 (2017).
14 Bernard-Valnet, R. et al. CD8 T cell-mediated killing of orexinergic neurons induces a narcolepsy-like phenotype in mice. Proc Natl Acad Sci USA 113, 10956-10961, doi:10.1073/pnas.1603325113 (2016).
15 Latorre, D. et al. T cells in patients with narcolepsy target self-antigens of hypocretin neurons. Nature 562, 63-68, doi:10.1038/s41586-018-0540-1 (2018).
16 Newell, E. W. et al. Combinatorial tetramer staining and mass cytometry analysis facilitate T-cell epitope mapping and characterization. Nat Biotechnol 31, 623-629, doi:10.1038/nbt.2593 (2013).
17 Su, L. F., Kidd, B. A., Han, A., Kotzin, J. J. & Davis, M. M. Virus-specific CD4(+) memory-phenotype T cells are abundant in unexposed adults. Immunity 38, 373-383, doi:10.1016/j.immuni.2012.10.021 (2013).
18 Bentzen, A. K. et al. Large-scale detection of antigen-specific T cells using peptide-MHC-1 multimers labeled with DNA barcodes. Nat Biotechnol 34, 1037-1045, doi:10.1038/nbt.3662 (2016).
19 Han, A., Glanville, J., Hansmann, L. & Davis, M. M. Linking T-cell receptor sequence to functional phenotype at the single-cell level. Nat Biotechnol 32, 684-692, doi:10.1038/nbt.2938 (2014).
20 Glanville, J. et al. Identifying specificity groups in the T cell receptor repertoire. Nature 547, 94-98, doi:10.1038/nature22976 (2017).
21 Ueno, T., Tomiyama, H., Fujiwara, M., Oka, S. & Takiguchi, M. Functionally impaired HIV-specific CD8 T cells show high affinity TCR-ligand interactions. J Immunol 173, 5451-5457 (2004).
22 Sibener, L. V. et al. Isolation of a Structural Mechanism for Uncoupling T Cell Receptor Signaling from Peptide-MHC Binding. Cell 174, 672-687 e627, doi:10.1016/j.cell.2018.06.017 (2018).
23 Rubio-Godoy, V. et al. Discrepancy between ELISPOT IFN-gamma secretion and binding of A2/peptide multimers to TCR reveals interclonal dissociation of CTL effector function from TCR-peptide/MHC complexes half-life. Proc Natl Acad Sci USA 98, 10302-10307, doi:10.1073/pnas.181348898 (2001).
24 Andersen, R. S. et al. Dissection of T-cell antigen specificity in human melanoma. Cancer Res 72, 1642-1650, doi:10.1158/0008-5472.CAN-11-2614 (2012).
25 Demachi-Okamura, A. et al. Epstein-Barr virus nuclear antigen 1-specific CD4⁺ T cells directly kill Epstein-Barr virus-carrying natural killer and T cells. Cancer science 99, 1633-1642, doi:10.1111/j.1349-7006.2008.00852.x (2008).
26 Jensen, K. K. et al. Improved methods for predicting peptide binding affinity to MHC class II molecules. Immunology 154, 394-406, doi:10.1111/imm.12889 (2018).
27 Siebold, C. et al. Crystal structure of HLA-DQ0602 that protects against type 1 diabetes and confers strong susceptibility to narcolepsy. Proc Natl Acad Sci USA 101, 1999-2004, doi:10.1073/pnas.0308458100 (2004).
28 Sakurai, T. et al. Orexins and orexin receptors: a family of hypothalamic neuropeptides and G protein-coupled receptors that regulate feeding behavior. Cell 92, 573-585 (1998).
29 Khodadoust, M. S. et al. Antigen presentation profiling reveals recognition of lymphoma immunoglobulin neoantigens. Nature 543, 723-727, doi:10.1038/nature21433 (2017).
30 Uchtenhagen, H. et al. Efficient ex vivo analysis of CD4⁺ T-cell responses using combinatorial HLA class II tetramer staining. Nature communications 7, 12614, doi:10.1038/ncomms12614 (2016).
31 Lo, W. L., Solomon, B. D., Donermeyer, D. L., Hsieh, C. S. & Allen, P. M. T cell immunodominance is dictated by the positively selecting self-peptide. eLife 3, e01457, doi:10.7554/eLife.01457 (2014).
32 Osborne, G. W., Andersen, S. B. & Battye, F. L. Development of a novel cell sorting method that samples population diversity in flow cytometry. Cytometry A 87, 1047-1051, doi:10.1002/cyto.a.22678 (2015).
33 Partinen, M. et al. Increased incidence and clinical picture of childhood narcolepsy following the 2009 H1N1 pandemic vaccination campaign in Finland. PLoS One 7, e33723, doi:10.1371/journal.pone.0033723 (2012).
34 Han, F. et al. Narcolepsy onset is seasonal and increased following the 2009 H1N1 pandemic in China. Ann Neurol 70, 410-417, doi:10.1002/ana.22587 (2011).
35 Le Nours, J. et al. Atypical natural killer T-cell receptor recognition of CD1d-lipid antigens. Nature communications 7, 10570, doi:10.1038/ncomms10570 (2016).
36 Li, M. O. & Flavell, R. A. TGF-beta: a master of all T cell trades. Cell 134, 392-404, doi:10.1016/j.cell.2008.07.025 (2008).
37 Brochet, X., Lefranc, M. P. & Giudicelli, V. IMGT/V-QUEST: the highly customized and integrated system for IG and TR standardized V-J and V-D-J sequence analysis. Nucleic Acids Res 36, W503-508, doi:10.1093/nar/gkn316 (2008).
38 Hua, L. et al. Cytokine-dependent induction of CD4⁺ T cells with cytotoxic potential during influenza virus infection. J Virol 87, 11884-11893, doi:10.1128/JVI.01461-13 (2013).
39 Vagaska, B. et al. MHC-class-II are expressed in a subpopulation of human neural stem cells in vitro in an IFNgamma-independent fashion and during development. Scientific reports 6, 24251, doi:10.1038/srep24251 (2016).
40 Block, M. L., Zecca, L. & Hong, J. S. Microglia-mediated neurotoxicity: uncovering the molecular mechanisms. Nature reviews. Neuroscience 8, 57-69, doi:10.1038/nrn2038 (2007).
41 Wan, X. et al. Pancreatic islets communicate with lymphoid tissues via exocytosis of insulin peptides. Nature, doi:10.1038/s41586-018-0341-6 (2018).
42 Risnes, L. F. et al. Disease-driving CD4⁺ T cell clonotypes persist for decades in celiac disease. J Clin Invest 128, 2642-2650, doi:10.1172/JC198819 (2018).
43 Newell, E. W. et al. Structural basis of specificity and cross-reactivity in T cell receptors specific for cytochrome c-I-E(k). J Immunol 186, 5823-5832, doi:10.4049/jimmunol.1100197 (2011).
44 Jiang, W. et al. pH-susceptibility of HLA-DO tunes DO/DM ratios to regulate HLA-DM catalytic activity. Scientific reports 5, 17333, doi:10.1038/srep17333 (2015).
45 Battye, T. G., Kontogiannis, L., Johnson, O., Powell, H. R. & Leslie, A. G. iMOSFLM: a new graphical interface for diffraction-image processing with MOSFLM. Acta Crystallogr D Biol Crystallogr 67, 271-281, doi:10.1107/S0907444910048675 (2011).
46 Evans, P. Scaling and assessment of data quality. Acta Crystallogr D Biol Crystallogr 62, 72-82, doi:10.1107/S0907444905036693 (2006).
47 McCoy, A. J. Solving structures of protein complexes by molecular replacement with Phaser. Acta Crystallogr D Biol Crystallogr 63, 32-41, doi:10.1107/S0907444906045975 (2007).
48 Emsley, P. & Cowtan, K. Coot: model-building tools for molecular graphics. Acta Crystallogr D Biol Crystallogr 60, 2126-2132, doi:10.1107/S0907444904019158 (2004).
49 Afonine, P. V. et al. Joint X-ray and neutron refinement with phenix.refine. Acta Crystallogr D Biol Crystallogr 66, 1153-1163, doi:10.1107/S0907444910026582 (2010).
50 Morin, A. et al. Collaboration gets the most out of software. eLife 2, e01456, doi:10.7554/eLife.01456 (2013).
51 Schrödinger, LLC. The PyMOL Molecular Graphics System, Version 2.0. (2015).
52 Sateia, M. J. International classification of sleep disorders-third edition: highlights and modifications. Chest 146, 1387-1394, doi:10.1378/chest.14-0970 (2014).

1. A method of detecting an autoreactive T cell specific for a hypocretin (HCRT) autoantigen, the method comprising:
a) obtaining a biological sample comprising T cells from a subject at risk of developing narcolepsy, suspected of having narcolepsy, or diagnosed with narcolepsy; and
b) detecting binding of the autoreactive T cell to an antigen presenting cell presenting at its surface the HCRT autoantigen in a complex with human leukocyte antigen (HLA)-DQ6, wherein the autoreactive T cell expresses a T cell receptor that specifically binds to the HCRT autoantigen.

2. The method of claim 1, wherein the HLA-DQ6 is encoded by a DQB1*0602 allele.

3. The method of claim 1, further comprising determining if the T cell receptor is encoded by a TRBV29-1_J2-5 gene and a TRAV6_J24 gene carrying a risk allele encoding TRAJ24L.

4. The method of claim 3, wherein said determining comprises sequencing a TCR alpha gene and a TCR beta gene of the autoreactive T cell, or performing an immunoassay with an anti-idiotype antibody that specifically binds to the T cell receptor encoded by the TRBV29-1 J2-5 gene and the TRAV6 J24 gene carrying the risk allele encoding TRAJ24L.

5. (canceled)

6. The method of claim 1, further comprising detecting activation of the autoreactive T cell.

7. (canceled)

8. The method of claim 1, wherein the HCRT autoantigen is a peptide fragment of a prepro-HCRT protein comprising an autoantigen epitope that specifically binds to the HLA-DQ6.

9-10. (canceled)

11. The method of claim 8, wherein the HCRT autoantigen comprises residues 1-13, residues 25-37, residues 56-69, residues 87-100, or residues 87-97 of an HCRT numbered relative to the reference sequence of SEQ ID NO:35.

12-15. (canceled)

16. The method of claim 8, wherein the autoantigen comprises an amino acid sequence selected from the group consisting of SEQ ID NOS:31-34.

17. The method of claim 8, wherein the C-terminus of the HCRT autoantigen is amidated.

18. The method of claim 1, wherein the antigen presenting cell is a dendritic cell, a macrophage, a microglial cell, or an artificial antigen presenting cell.

19-20. (canceled)

21. The method of claim 1, wherein the biological sample is blood, peripheral blood mononuclear cells, lymphoid tissue, cerebrospinal fluid, or nervous system tissue.

22. The method of claim 1, wherein the autoreactive T cell is a CD4′ T cell.

23. The method of claim 1, further comprising diagnosing the subject with narcolepsy by comparing the number of autoreactive T cells specific for the HCRT autoantigen in the biological sample to a reference value for the number of autoreactive T cells specific for the HCRT autoantigen in a control sample isolated from a healthy donor, wherein the subject is diagnosed as having narcolepsy if the number of the autoreactive T cells specific for the HCRT autoantigen is higher in the biological sample than the reference value.

24. The method of claim 23, further comprising detecting activation or expansion of autoreactive T cells specific for the HCRT autoantigen that have a risk allele encoding TRAJ24L in vivo to confirm a diagnosis of narcolepsy.

25. The method of claim 23, further comprising treating the subject for narcolepsy if the subject is diagnosed with narcolepsy based on the number of the autoreactive T cells specific for the HCRT autoantigen in the biological sample.

26-27. (canceled)

28. The method of claim 1, wherein the subject has type 1 narcolepsy.

29. A method of diagnosing and treating narcolepsy, the method comprising:
a) obtaining a biological sample comprising T cells from a subject at risk of developing narcolepsy, suspected of having narcolepsy, or diagnosed with narcolepsy;
b) detecting binding of a T cell to an antigen presenting cell presenting at its surface an HCRT autoantigen in a complex with human leukocyte antigen (HLA)-DQ6, wherein the T cell expresses a T cell receptor that specifically binds to the HCRT autoantigen;
c) diagnosing the subject by determining if the T cell receptor is encoded by a TRBV29-1_J2-5 gene and a TRAV6_J24 gene carrying a risk allele encoding TRAJ24L, wherein detection of the TRBV29-1_J2-5 gene and the TRAV6_J24 gene carrying the risk allele encoding TRAJ24L indicates that the subject has narcolepsy; and
d) treating the subject for narcolepsy if the subject is diagnosed with narcolepsy based on the detection of the TRBV29-1_J2-5 gene and the TRAV6_J24 gene carrying the risk allele encoding TRAJ24L.

30. The method of claim 29, wherein said determining if the T cell receptor is encoded by a TRBV29-1_J2-5 gene and a TRAV6_J24 gene carrying the risk allele encoding TRAJ24L comprises sequencing the TCR alpha gene and the TCR beta gene of the T cell bound to the antigen presenting cell, or performing an immunoassay with an anti-idiotype antibody that specifically binds to the T cell receptor encoded by the TRBV29-1 J2-5 gene and the TRAV6 J24 gene carrying the risk allele encoding TRAJ24L.

31. (canceled)

32. The method of claim 29, wherein said treating comprises administering a central nervous system stimulant, a norepinephrine reuptake inhibitor (NRI), a selective serotonin reuptake inhibitor, a tricyclic antidepressant, sodium oxybate, or a combination thereof.

33. The method of claim 29, further comprising detecting activation or expansion of autoreactive T cells specific for the HCRT autoantigen that have a risk allele encoding TRAJ24L in vivo to confirm a diagnosis of narcolepsy.

34. An isolated hypocretin (HCRT) autoantigen peptide selected from the group consisting of:
a) an HCRT autoantigen peptide comprising an amino acid sequence selected from the group consisting of SEQ ID NOS:31-34; and
b) an HCRT autoantigen peptide comprising an amino acid sequence having at least 70% identity to an amino acid sequence selected from the group consisting of SEQ ID NOS:31-34, wherein the HCRT autoantigen peptide comprises an HCRT autoantigen T cell epitope that binds to a T cell receptor encoded by a TRBV29-1_J2-5 gene and a TRAV6_J24 gene carrying a risk allele encoding TRAJ24L.

35-40. (canceled)
